(19) United States
(12) Patent Application Publication
(10) Pub. No.: US 2006/0034782 A1
(43) Pub. Date: Feb. 16, 2006

(54) Biofilm therapy interproximal devices

(76) Inventors: Dale G. Brown, Wharton, TX (US); Ira D. Hill, Austin, TX (US); Michael R. Schweigert, Missouri City, TX (US)

Correspondence Address: BANNER & WITCOFF, LTD., 28 STATE STREET, 28th FLOOR, BOSTON, MA, 02109-9601, US

(21) Appl. No.: 11/196,827

(22) Filed: Aug. 3, 2005

Related U.S. Application Data
(63) Continuation-in-part of application No. 10/005,902, filed on Dec. 4, 2001.
(63) Continuation-in-part of application No. 10/331,800, filed on Dec. 30, 2002.
(63) Continuation-in-part of application No. 10/073,682, filed on Feb. 11, 2002.
(63) Continuation-in-part of application No. 10/334,089, filed on Dec. 30, 2002.

(51) Int. Cl. A61C 5/00; A61K 8/81

(52) U.S. Cl. 424/49; 433/217.1

(57) ABSTRACT

The present invention discloses and claims various interproximal devices and associated methods for: (a) removing and disrupting interproximally, supragingival and subgingivally the microbiological burden associated with biofilms, (b) controlling biofilm influence among at risk adults on certain systemic chronic diseases including: Type II diabetes, heart disease, atherosclerosis, myocardial infarction and osteoporosis, and (c) maintaining periostasis in at risk adults.

### CROSS-REFERENCE TO RELATED APPLICATIONS

This application is a continuation-in-part of the following copending applications: U.S. patent application Ser. No. 10/005,902, filed Dec. 4, 2001 entitled “Biofilm Therapy”; U.S. patent application Ser. No. 10/331,800, filed Dec. 30, 2002, entitled, “Coated Micromesh Dental Devices Overcoated with Imbedded Particulate”; U.S. patent application Ser. No. 10/073,682, filed 11 Feb. 2002, entitled, “Micromesh Interproximal Devices”; and U.S. patent application Ser. No. 10/334,089, filed Dec. 30, 2002, entitled, “Particulate Coated Monofilament Devices. The disclosures of these applications are hereby incorporated herein by reference.

### BACKGROUND OF THE INVENTION

Dental floss is defined in Webster's New World Dictionary, 1983, as “ . . . thread for removing food particles between the teeth.”

The concept of using dental floss for cleansing interproximal spaces appears to have been introduced by Parmly in 1819, Practical Guide to the Management of Teeth, Cullins & Croft Philadelphia, Pa. Numerous types of floss were developed and used for cleaning interproximal and subgingival surfaces, until finally in 1948 Bass established the optimum characteristics of dental floss, Dental Items of Interest, 70, 921-34 (1948).

Bass cautioned that dental floss treated with sizing, binders and/or wax produces a “cord” effect as distinguished from the desired “spread filament effect”. This cord effect reduces flossing efficiency dramatically and visually eliminates splaying (i.e., the flattening and spreading out of filaments) necessary to achieve the required interproximal and subgingival mechanical cleaning. This cleaning is then required to be followed by the entrapment and removal of loosened: debris, plaque and microscopic materials from interproximal spaces by the “spread” floss as it is removed from between teeth.

Proper use of dental floss is necessary to clean the considerable surface area on the interproximal surfaces of teeth (approximately 40% of total tooth surfaces), which cannot usually be reached by other cleaning methods or agents, e.g., the bristles of a toothbrush, the swishing action of a rinse, or by the pulsating stream from an oral irrigator.

Historically, the purpose of dental floss was to:
(1) dislodge and remove any decomposing food material, debris, etc., that has accumulated at the interproximal surfaces, which could not be removed by other oral hygiene means, and
(2) dislodge and remove as much as possible the growth of bacterial material (plaque, tartar, calculus . . . eventually to be classified as biofilm) that had accumulated there since the previous cleaning.

Effective oral hygiene requires that three control elements be maintained by the individual:
(1) Physical removal of stains, plaque and tartar. This is accomplished in the strongest sense by scraping and abrasion in the dentist's office during prophylaxis, scaling or root planing. Self administered procedures are required frequently between visits to the oral care professional and range from tooth brushing with an appropriate abrasive toothpaste through flossing and water jet action down to certain abrasive foods and even the action of the tongue against tooth surfaces.
(2) Surfactant Cleaning. This is required to remove: food debris and staining substances before they adhere to the tooth surface; normal dead cellular (epithelial) material which is continually sloughed off from the surfaces of the oral cavity and microbial degradation products derived from all of the above. Research has shown that the primary source of bad breath is the retention and subsequent degradation of dead cellular material sloughed off continuously by the normal, healthy mouth. Besides the obvious hygienic and health benefits related to simple cleanliness provided by surfactants, there is an important cosmetic and sense-of-well-being benefit provided by surfactant cleansing.
(3) Frequency of Cleansing. This is perhaps the most difficult to provide in today's fast-paced work and social environment. Most people recognize that their teeth should be brushed at least 3 times a day and flossed at least once a day. The simple fact is that most of the population brush once a day, some brush morning and evening, but precious few carry toothbrush and dentifrice to use the other three or four times a day for optimal oral hygiene. Consumer research suggests that the population brushes an average of about 1.3 times a day. Most surprising, less than 15% of adults floss regularly. Reasons offered for not flossing: difficult to do, painful, not effective, doesn't seem to do anything, and leaves a bad taste. There is generally no appreciation for the role plaque (biofilm) buildup plays in exacerbating chronic diseases such as Type II diabetes, heart disease, etc.

Until the introduction of micromesh dental floss as described in copending U.S. patent application Ser. No. 10/073,682, entitled, “Micromesh Interproximal Devices”; there have been two types of interproximal devices available commercially: multifilament dental flosses and monofilament dental tapes.

Examples of multifilament dental flosses are described in the following U.S. Pat. Nos., which are hereby incorporated by reference:

| | | | | | |
|---|---|---|---|---|---|
| 4,911,927; | 4,029,113; | 4,610,872; | 4,034,771; | 5,908,039; | 2,667,443; |
| 3,830,246; | 1,149,376; | 1,069,874; | 5,830,495; | 2,748,781; | 1,138,479; |
| 1,839,486; | 1,943,856; | 6,080,481; | 2,700,636; | 3,699,979; | 3,744,499; |
| 3,837,351; | 4,414,990; | 3,330,732; | 5,967,155; | 5,937,874; | 5,505,216; |
| 5,503,842; | 5,032,387; | 4,950,479; | 5,098,711; | 1,989,895; | 5,033,488; |
| 2,542,518; | 2,554,464; | 1,285,988; | 1,839,483; | 4,151,851; | 2,224,489; |
| 2,464,755; | 2,381,142; | 3,800,812; | 3,830,246; | 3,897,795; | 3,897,796; |
| 4,215,478; | 4,033,365; | 3,771,536; | 3,943,949; | 6,016,816; | 6,026,829; |
| 5,353,820; | 5,557,900; | 5,226,435; | 5,573,850; | 5,560,377; | 5,526,831; |
| 5,423,337; | 5,220,932; | 4,548,219; | 3,838,702; | 5,904,152; | 4,911,927; |
| 5,711,935; | 5,165,913; | and | 5,098,711. | | |

Examples of monofilament dental tapes are described in the following U.S. Pat. Nos., which are hereby incorporated by reference:

| | | | | | |
|---|---|---|---|---|---|
| Re. 35,439; | 3,800,812; | 4,974,615; | 5,760,117; | 5,433,226; | 5,479,952; |
| 5,503,842; | 5,755,243; | 5,845,652; | 5,884,639; | 5,918,609; | 5,962,572; |
| 5,998,431; | 6,003,525; | 6,083,208; | 6,198,830; | 6,161,555; | 6,027,192; |
| 5,209,251; | 5,033,488; | 5,518,012; | 5,911,228; | 5,220,932; | 4,776,358; |
| 5,718,251; | 5,848,600; | 5,787,758; | and | 5,765,576. | |

It is generally accepted that both monofilament and multifilament dental flosses are not “user-friendly” products, i.e., flossing with either is difficult to do. Flossing is generally associated with pain and bleeding and it results in a bad taste in the mouth. Most market researchers agree that anything that can be done to make flossing more positive should be implemented to encourage more frequent flossing and more wide spread floss and/or tape use. The addition to floss and tape of: full spectrum flavor oils, mouth conditioning substances such as silicones along with cleaners and abrasives that are perceived as “working” as taught by the copending Patent Applications: “Coated Multifilament Dental Devices Overcoated with Imbedded Particulate” and “Coated Monofilament Dental Devices Overcoated with Imbedded Particulate” are all sources of positive feed back to the flosser that would be considered encouraging and supportive, e.g., “it's doing something.” To achieve these with micromesh dental floss requires basic changes in present micromesh floss manufacturing.

Most commercial monofilament and multifilament interproximal devices marketed at the present time contain various coatings of wax or wax like substances that function as: (1) binders for the various multifilament flosses to minimize fraying, (2) lubricants, (3) flavor carriers, and/or (4) fluoride carriers for both monofilament and multifilament devices.

An almost universal shortcoming common to most waxed multifilament dental flosses and monofilament tapes is the user perception during flossing that the dental floss or dental tape is “not working” and/or “not cleaning”, etc.

In fact, most of these devices have only marginal efficacy with respect to removing biofilms (plaque). Biofilms generally require physical abrasive-type action to be effectively removed. Periodic professional cleaning is a recommended means for effectively controlling biofilm formation.

From 1960 thru 1982, numerous clinical studies reported that there is no clinical difference as to plaque removal and gingivitis scores between waxed and unwaxed multifilament dental floss. Note, both are “cord” flosses and contain sizing, binders, etc. These studies also confirmed that waxed and unwaxed floss are approximately 50% effective with respect to plaque removal and gingivitis scores. Thus the “cord” effect severely restricts efficiency of flossing and especially physical abrasive-type action associated with multifilament flosses that splay as described by Bass.

O'Leary in 1970, and Hill et al. in 1973, found no difference in the interproximal cleansing properties of waxed and unwaxed dental floss. This was reconfirmed in 1982 by Lobene et al. who showed no significant clinical difference on plaque and gingivitis scores. Similar results, i.e., no clinical difference between waxed and unwaxed multifilament dental floss with respect to reduced gingival inflammation were shown by Wunderlich in 1981. No differences in plaque removal were reported by Schmidt et al. in 1981 with multifilament flosses of various types. Stevens, 1980, studied multifilament dental floss with variable diameters and showed no difference in plaque and gingival health. Carter et al. 1975, studied professional and self administered waxed and unwaxed multifilament dental floss, both significantly, reduced gingival bleeding of interproximal and gingival sulci. Unwaxed multifilament dental floss appeared slightly, but not significantly more effective.

In view of this clinical work, it is not surprising that most of the multifilament dental floss sold today is, contrary to the teaching of Bass, bonded and/or waxed. The “bonding” in the yarn industry today is used more to facilitate processing and production during multifilament dental floss manufacture and packaging than for “flossing” reasons. Since clinical tests show no difference between waxed and unwaxed multifilament dental floss (both unfortunately are “bonded”), the multifilament dental floss industry has been comfortable with the yarn industry's propensity to use bonding agents in multifilament dental floss, thereby sacrificing splaying and physical abrasive-type cleaning. Of course, monofilament dental tapes do not splay and have a basic shortcoming with respect to abrasive-type cleaning.

The development of micromesh dental flosses, which combine the strengths and advantages of multifilament dental flosses and monofilament dental tapes, while minimizing the shortcomings of monofilament and multifilament devices, is described in detail in copending U.S. patent application Ser. No. 10/073,682, entitled “Micromesh Interproximal Devices”.

The classification of plaque as a biofilm is considered a major advance in the development of more effective “self-treatment” oral care products. See the following biofilm references:

Greenstein and Polson, J. Periodontol., May 1998, 69:5:507-520; van Winkelhoff, et al., J. Clin. Periodontol., 1989, 16:128-131; and Wilson, J. Med. Microbiol., 1996, 44:79-87.
Biofilms are defined as “ . . . matrix-enclosed bacterial population adherent to each other and to the surface or intersurfaces. These masses secrete an exopolysaccharide matrix for protection. Considerably higher concentrations of drugs are needed to kill bacteria in biofilms than organisms in aqueous suspensions.”

Costerton, J. W., Lewandowski, Z., DeBeer, D., Caldwell, D., Korber, D., James, G. Biofilms, the customized microniche. J. Bacterio., 1994, 176:2137-2142.
The unique attributes of biofilms are being recognized as increasingly important in the 1990's. Future studies into the mode of growth of biofilms will allow manipulation of the bacterial distribution.

Douglass, C. W., Fox, C. H. Cross-sectional studies in periodontal disease: Current status and implications for dental practice. Adv. Dent. Res., 1993, 7:26-31.

Greenstein, G. J., Periodontal response to mechanical non-surgical therapy: A review. Periodontol., 1992, 63:118-130.
Mechanical therapy remains effective with caveats to compliance and skill of therapists.

Marsh, P. D., Bradshaw, D. J. Physiological approaches to the control of oral biofilms. Adv. Dent. Res., 1997, 11:176-185.
Most laboratory and clinical findings support the concept of physiological control. Further studies will reveal details of biofilm diversity.

Page, R. C., Offenbacher, S., Shroeder, H., Seymour, G. J., Kornman, K. S., Advances in the pathogenesis of periodontitis: Summary of developments, clinical implications and future directions. Periodont. 2000, 1997, 14:216-248.
Genetic susceptibility to three oral anaerobic bacteria play an important part in the progression of periodontitis. Acquired and environmental risk factors exacerbate the problem. Mechanical disruption will remain an effective and essential part of periodontal therapy. (emphasis added)

Papapanou, P. N., Engebretson, S. P., Lamster, I. B. Current and future approaches for diagnosis of periodontal disease. NY State Dent. J., 1999, 32-39.
New techniques are available such as a novel pocket depth measurement device, microscopic techniques, immunoassay, DNA probes, BANA hydrolysis tests. These more clearly define the nature of periodontitis.

The classification of plaque as a biofilm calls for more effective interproximal devices, with respect to removing, disrupting and/or controlling biofilms which requires: (a) physical particulate-abrasive-type cleaning interproximally and subgingivally when flossing, (b) chemotherapeutic (topical, antimicrobial) treatment of residual biofilm remaining after flossing. Such physical-abrasive cleaning is not available from commercial multifilament and monofilament interproximal devices marketed today.

### SUMMARY OF THE INVENTION

The present invention discloses and claims various interproximal devices and associated methods for: (a) removing and disrupting interproximally, the supragingival and subgingival microbiological burden associated with biofilms, (b) maintaining periostasis in at risk adults, and (c) controlling biofilm influence on certain systemic chronic diseases among at risk adults, including: Type II diabetes, heart disease, atherosclerosis, myocardial infarction and osteoporosis.

Micromesh dental floss is described in the referenced Patent Application, entitled “Micromesh Interproximal Devices” as a random: net, web or honeycomb-type integrated structure as distinguished from the more orderly monofilament and multifilament or woven structures used heretofore for interproximal devices. These micromesh structures are produced at low cost by integrating a rotating fibrillator device into a flat stretched film or tape producing operation, such as described in U.S. Pat. No. 5,578,373. A wide range of fibrillators are available to produce an almost endless array of micromesh structures including those illustrated in FIGS. 1a through 1f and further shown in FIGS. 2 through 4. All of these are suitable for use as particulate overcoated coated micromesh interproximal devices of the present invention.

The present invention is directed to biofilm-responsive, coated micromesh dental flosses containing an antimicrobial and overcoated with soft abrasives which:
(a) are suitable for physical-abrasive-type removal and disruption of biofilms that form on interproximal, supragingival and subgingival tooth surfaces not reachable by brushing or rinsing;
(b) topically treat residual biofilm remaining interproximally after flossing with an antimicrobial to chemotherapeutically treat the microflora in the residual biofilm to maintain periostasis among at risk adults;
(c) control antimicrobial-based tooth staining; and
(d) physically entrap from interproximal sites: loosened biofilm, debris, food particles and materia alba.

The coated micromesh dental flosses of the present invention containing an antimicrobial are overcoated with an imbedded particulate abrasive that remains imbedded in the micromesh floss, saliva soluble, base coating until said base coating in which it is imbedded is eventually released from the micromesh substrate during flossing.

During flossing, at the outset, the imbedded particulate abrasive overcoating functions as a “soft” abrasive version of an oral-type sandpaper removing and disrupting biofilms and antimicrobial stained pellicle. Essentially the first pass through an interproximal space by the imbedded particulate, overcoated, micromesh dental floss results in a gentle “sandpaper” abrasive effect on the biofilms present, which effect is eventually followed by dissolving and/or breaking up of the saliva soluble base coating containing the particulate abrasive which is present on the micromesh net.

After the saliva soluble base coating is released, the soft abrasive particulate overcoating works in conjunction with the micromesh net interproximally to continue to remove and disrupt biofilms until the particulate abrasive is flushed away and/or dissolved by saliva. That is, the released particulate abrasive cooperates with the fibrillated micromesh dental floss as the floss is being worked over interproximal, supragingival and subgingival surfaces to continue to deliver physical-abrasive-type cleaning and disruption of those biofilms formed on interproximal, supragingival and subgingival tooth surfaces.

The physical-abrasive-type cleaning and disruption of biofilms achieved with the various imbedded particulate soft abrasives overcoated micromesh dental flosses of the present invention continues until:
(a) the micromesh dental floss is removed from the space and flossing of the area is discontinued,
(b) the particulate abrasive dissolves and/or is washed away by saliva, and/or
(c) the biofilm is physically removed or disrupted.

The physical-abrasive-type cleaning and disruption of biofilms with the imbedded particulate abrasive overcoated micromesh dental flosses of the present invention are simultaneously supplemented with a chemotherapeutic treatment by various chemotherapeutic, antimicrobial substances contained in: (1) the base coating, (2) the particulate abrasive, and/or (3) other particulate overcoating substances used to introduce flavor, mouth feel, etc., attributes into the particulate overcoated micromesh dental flosses of the invention. In the latter version, these chemotherapeutic substances are released onto interproximal tooth surfaces during flossing along with the saliva soluble particulate that releases from the base coating to help disrupt and control the microflora associated with residual biofilm not removed during flossing.

Surprisingly, the particulate abrasive overcoating imbedded in the base coating on the micromesh dental floss of the present invention exhibits unexpected gentleness along with lower than expected abrasivity which, for purposes of the present invention, allows more abrasive particulates to be used in the overcoating, such as pumice, alumina, silica, etc. This “soft abrasive” effect is attributed in part to the cushion effect contributed by the saliva soluble base coating to the imbedded particulate abrasive. That is, the base coating containing the partially imbedded particulate abrasive tends to cushion the impact of the exposed portion of the abrasive particulate onto tooth surfaces during flossing. See FIGS. 10, 16 and 17. When the abrasive/saliva soluble coating mixture breaks free from the micromesh during flossing, the base coating tends to help lubricate the particulate abrasive/micromesh combination further reducing the abrasivity of the particulate soft abrasive on tooth surfaces.

Accordingly, one embodiment of the present invention comprises biofilm-responsive, antimicrobial, micromesh dental floss devices suitable for maintaining periostasis among at risk adults.

A further embodiment of the present invention comprises saliva soluble coated micromesh dental floss devices containing a releasable antimicrobial with particulate soft abrasives imbedded in the coating, thereby rendering the floss biofilm-responsive during and after flossing and suitable for maintaining periostasis among at risk adults.

Another embodiment of the invention comprises a self-treatment means for routinely removing and disrupting biofilms formed on interproximal, supragingival and subgingival tooth surfaces, and for antimicrobially treating residual biofilms that remain interproximally after flossing, thereby maintaining periostasis among at risk adults.

Still another embodiment of the invention comprises a method for overcoating saliva soluble, coated, antimicrobial, micromesh dental flosses with imbedded particulate abrasives of various particle sizes and particle size distributions as a means for effectively removing and disrupting biofilms and antimicrobial stains from interproximal tooth surfaces.

Yet another embodiment of the invention comprises a patient self-treatment method for periodically removing and disrupting biofilms that form on interproximal, supragingival and subgingival tooth surfaces, while treating residual biofilms with an antimicrobial to maintain periostasis among at risk adults.

A further embodiment of the invention comprises biofilm-responsive, antimicrobial, micromesh dental devices overcoated with imbedded particulate abrasives and containing a releasable saliva soluble base coating which contains an antimicrobial suitable for maintaining periostasis, while simultaneously removing antimicrobial stains from interproximal tooth surfaces.

Another embodiment of the invention comprises biofilm-responsive, antimicrobial, micromesh dental devices overcoated with active imbedded particulate soft abrasives suitable for maintaining periostasis among at risk adults.

Still another embodiment of the invention comprises biofilm-responsive, antimicrobial, micromesh dental devices overcoated with soft abrasives suitable for maintaining periostasis among at risk adults, where the soft abrasives include: silica, pumice, alumina, calcium carbonate and dicalcium phosphate dihydrate.

Yet another embodiment of the invention comprises biofilm-responsive, antimicrobial, micromesh dental devices suitable for maintaining periostasis among at risk adults, overcoated with imbedded, particulate, soft abrasives, where said abrasives contain other substances ranging from flavorants, antimicrobials and cleaning substances to mouth conditioners and various pharmaceutical substances.

A further embodiment of the invention comprises improved antimicrobial, micromesh dental flosses suitable for maintaining periostasis with an overcoating of imbedded, particulate, soft abrasive.

Still another embodiment of the invention comprises improved antimicrobial, micromesh dental flosses suitable for maintaining periostasis with overcoatings of imbedded, particulate, soft abrasive and saliva soluble particulate substances containing flavorant and mouth conditioning substances.

Another embodiment of the invention comprises improved antimicrobial, micromesh dental devices suitable for maintaining periostasis with an overcoating of imbedded, particulate, soft abrasives containing a saliva soluble substance with flavorants, mouth conditioners and tartar control agents.

Yet another embodiment of the invention comprises a method for improving micromesh dental flosses with saliva soluble coatings containing antimicrobials, suitable for maintaining periostasis comprising sequential overcoating of said saliva soluble base coated, antimicrobial, micromesh dental flosses with two or more particulates having substantially different densities, wherein said various particulates are imbedded into said base coating prior to cooling and solidifying.

Still another embodiment of the invention comprises improved commercial, emulsion coated, antimicrobial, micromesh dental floss with an overcoating of imbedded, particulate, soft abrasive suitable for maintaining periostasis.

Another embodiment of the invention comprises improved saliva soluble, coated, extensively fibrillated, micromesh dental floss suitable for maintaining periostasis containing an antimicrobial with an overcoating of imbedded, particulate, soft abrasive.

Still another embodiment of the invention comprises interproximal devices and associated methods for: (a) removing, disrupting and controlling interproximally, the supragingival and subgingival microbiological burden associated with biofilms, and (b) maintaining and controlling periostasis influence on certain systemic chronic diseases including: Type II diabetes, heart disease, atherosclerosis, myocardial infarction and osteoporosis.

Yet another object of the invention comprises an interproximal device suitable for treating various biofilm supported, chronic conditions selected from the group consisting of: Type II diabetes mellitus, atherosclerosis, heart disease, osteoporosis, HIV, myocardial infarction, and combinations thereof, comprising flossing regularly with an interproximal device comprising a fibrillated, high molecular weight polyethylene tape that is compression coated with a saliva soluble coating containing an antimicrobial and overcoated with a soft abrasive overcoating, wherein during flossing, said device:
(a) physically removes and disrupts interproximal biofilms,
(b) chemotherapeutically disrupts and controls residual interproximal biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba.

Another object of the invention comprises a method for treating various biofilm-supported, chronic conditions selected from the group consisting of: Type II diabetes mellitus, atherosclerosis, heart disease, osteoporosis, HIV, myocardial infarction, and combinations thereof, comprising flossing regularly with an interproximal device comprising a fibrillated, high molecular weight polyethylene tape that is compression coated with a saliva soluble coating containing a substantive antimicrobial and overcoated with a soft abrasive overcoating, wherein during flossing, said device:
(a) physically removes and disrupts interproximal biofilms,
(b) chemotherapeutically disrupts and controls residual interproximal biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba, and removing said spent device from interproximal spaces.

Still another object of the invention comprises an interproximal device suitable for reducing and controlling biofilm-supported glycated hemoglobin levels of Type II diabetics, comprising flossing regularly with an interproximal device comprising a fibrillated, high molecular weight polyethylene tape that is compression coated with a saliva soluble coating containing chlorhexidine digluconate and overcoated with soft abrasive overcoating, wherein during flossing, said device:
(a) physically removes and disrupts interproximal biofilms,
(b) chemotherapeutically disrupts and controls residual interproximal biofilms remaining after flossing by topically releasing the antimicrobial, chlorhexidine digluconate, onto said residual biofilms, thereby maintaining periostasis,
(c) controls chlorhexidine-based tooth staining, and
(d) physically entraps: loosened biofilm, debris, food particles and materia alba.

A further object of the invention comprises a method for reducing and controlling biofilm-supported glycated hemoglobin levels of Type II diabetics, comprising flossing regularly with an interproximal device comprising a fibrillated, high molecular weight polyethylene tape that is compression coated with a saliva soluble coating containing chlorhexidine digluconate and overcoated with soft abrasives overcoating, wherein during flossing, said device:
(a) physically removes and disrupts interproximal biofilms,
(b) chemotherapeutically disrupts and controls residual interproximal biofilms remaining after flossing by topically releasing chlorhexidine digluconate onto said residual biofilms, thereby maintaining periostasis,
(c) controls chlorhexidine-based tooth staining, and
(d) physically entraps: loosened biofilm, debris, food particles and materia alba, and removing said spent device from interproximal spaces.

Yet another object of the invention comprises an interproximal device suitable for controlling biofilm-supported carotid artery intima media thickness associated with increased risk of heart disease, comprising flossing regularly with an interproximal device comprising a fibrillated, high molecular weight polyethylene tape that is compression coated with a saliva soluble coating containing chlorhexidine digluconate and overcoated with soft abrasives overcoating, wherein during flossing, said device:
(a) physically removes and disrupts interproximal biofilms,
(b) chemotherapeutically disrupts and controls residual interproximal biofilms remaining after flossing by topically releasing chlorhexidine digluconate onto said residual biofilms, thereby maintaining periostasis,
(c) controls chlorhexidine-based tooth staining, and
(d) physically entraps: loosened biofilm, debris, food particles and materia alba.

Another object of the invention comprises a method suitable for controlling biofilm-supported carotid artery intima media thickness associated with increased risk of heart disease, comprising flossing regularly with an interproximal device comprising a fibrillated, high molecular weight polyethylene tape that is compression coated with a saliva soluble coating containing chlorhexidine digluconate and overcoated with soft abrasives overcoating, wherein during flossing, said device:
(a) physically removes and disrupts interproximal biofilms,
(b) chemotherapeutically disrupts and controls residual interproximal biofilms remaining after flossing by topically releasing chlorhexidine digluconate onto said residual biofilms, thereby maintaining periostasis,
(c) controls chlorhexidine-based tooth staining, and
(d) physically entraps: loosened biofilm, debris, food particles and materia alba, and removing said spent device from interproximal spaces.

For purposes of describing the present invention, the following terms are defined as set out below:

“Periostasis” defines a stabilized gingival condition, identified with at risk adults, where biofilm triggered gum disease, including: gingival detachment, bleeding gums and periodontal disease, as well as biofilm bacteria-based exacerbation of chronic system conditions, such as: Type II diabetes, cardiovascular disease, atherosclerosis, myocardial infarction, osteoporosis and low birth weight babies, are abated between regular visits to an oral care professional.

at risk defines those adults who have one or more chronic diseases which they regularly treat with medicine.

The terms “fiber” and “filament” are used synonymously throughout this specification in a manner consistent with the first three definitions of “fiber” and the first definition of “filament” as given in the New Illustrated Webster's Dictionary, ©1992 by J. G. Ferguson Publishing Co. the relevant disclosure of which is hereby incorporated herein by reference.

“Base coatings” for the micromesh dental devices are defined as those saliva soluble substances that coat micromesh dental devices for purposes of: lubrication and ease of floss insertion for carrying antimicrobials flavors and other additives, providing “hand” so the device can be wound around the fingers, etc., such as described in detail in Tables 3 to 4 below. These saliva soluble coatings generally comprise from about 25 to about 100% by weight of the micromesh floss.

Preferred saliva soluble, base coatings include:

(a) those emulsion coatings described in the following U.S. Pat. Nos., 4,950,479; 5,032,387; 5,538,667; 5,561,959; and 5,665,374, which are hereby incorporated by reference,

(b) various dental floss coatings, such as described in U.S. Pat. Nos. 5,908,039; 6,080,495; 4,029;113; 2,667,443; 3,943,949; 6,026,829; 5,967,155 and 5,967,153, which are hereby incorporated by reference, and

(c) those saliva soluble coatings described and claimed in co-pending U.S. patent applications Ser. Nos. 09/935,922; 09/935,920; 09/935,921 and 09/935,710, all filed on Aug. 23, 2001, which are hereby incorporated by reference.

All of the foregoing base coatings contain biofilm-responsive levels of one or more antimicrobials suitable for maintaining periostasis.

“Antimicrobial” includes various active ingredients that: control, disrupt and/or kill various microbiota associated with residual biofilms, which remain on tooth surfaces after flossing with the interproximal devices of the present invention. These include topical antimicrobials, such as: chlorhexidine digluconate (chlorhexidine), triclosan, benzylalkonium chloride, cetylpyridinium chloride, iodine, metronidazole and microbially active essential oils, such as thymol, menthol, etc.

“Particulate abrasives” are defined as saliva soluble, semi-soluble and insoluble abrasive substances having a wide range of particle sizes and particle size distribution that are effective in physically removing, disrupting and controlling biofilms, when imbedded into the saliva soluble, coated, micromesh devices of the present invention.

Preferred particulate abrasives include various insoluble inorganics such as glass beads, and various insoluble organics such as particles of polyethylene, polypropylene, etc.

Particularly preferred inorganic particulate abrasives include various: (1) insoluble dental abrasives such as: pumice, silica, alumina, silicon dioxide, magnesium oxide, aluminum hydroxide, diatomaceous earth, sodium potassium aluminum silicate, zirconium silicate, calcium silicate, fumed silica, hydrated silica, and (2) soluble dental abrasives such as: dicalcium phosphate dihydrate, anhydrous dicalcium phosphate, sodium tripolyphosphate, calcium carbonate, etc. See also Table 1 below.

Particularly preferred “active” particulate abrasives include peroxides such as: carbamide peroxide, calcium peroxide, sodium perborate, sodium percarbonate, magnesium peroxide, sodium peroxide, etc.; phosphates such as: sodium hexametaphosphate, tricalcium phosphate, etc.; and pyrophosphates such as: tetrasodium pyrophosphate, tetrapotassium pyrophosphate, sodium acid pyrophosphate, calcium pyrophosphate, etc. See also Table 2 below.

See also the following relevant U.S. Pat. Nos. 6,221,341; 3,491,776; 3,330,732; 3,699,979; 2,700,636; 5,220,932; 4,776,358; 5,718,251; 5,848,600; 5,787,758; and 5,765,576, which describe various oral care abrasives suitable for the present invention and are incorporated herein by reference.

“Releasable” particulate abrasive is defined as the property whereby a particulate abrasive, which is imbedded into the saliva soluble base coating on micromesh dental floss, remains substantive to said base coating until flossing begins, after which time the imbedded particulate abrasive in the base coating eventually separates from the micromesh along with the base coating which eventually dissolves and releases the particulate abrasive into saliva. Thus, the particulate abrasive remains available interproximally and subgingivally to work with the fibrillated micromesh floss, responding to biofilms encountered on interproximal, supragingival and subgingival tooth surfaces with physical-abrasive-type cleaning.

“Particulate abrasive load” is defined as the percent by weight of imbedded particulate abrasive contained on the coated micromesh dental device as a percent by weight of the device. See Tables 1, 2, 3 and 5 below.

“Base coat micromesh device load” is defined as the percent by weight of the base coating contained on the micromesh device as a percent by weight of the coated micromesh device.

“Total coating load” is defined as the percent by weight of the base coating plus the particulate abrasive overcoating imbedded in said coating on the micromesh device as a percent by weight of the device.

“Perceived Abrasive Factor (PAF)” is defined as the subjective level of perceived abrasivity when:
(1) winding the coated micromesh device with imbedded particulate abrasive around the fingers (i.e., “hand”), and
(2) when working the device across tooth surfaces with a sawing action.

PAF grades range from 0 through 4, i.e., imperceptible (0), slightly perceptible (1), perceptible (2), very perceptible (3) and very abrasive (4). See Tables 1, 2 and 9 below. PAF values of about 2 or greater are preferred. PAF values above 3 are particularly preferred. Permanent abrasives generally exhibit higher PAF values than releasable abrasives.

“Incidental Release Factor (IRF)” is defined as the percent by weight of the particulate abrasive retained on the coated micromesh dental device, when an 18 inch piece of the device is removed from a dispenser and wrapped around two fingers prior to flossing. (See Tables 1, 2 and 9.) IRF values over 90% reflect the degree to which the particulate abrasives are imbedded in the base coating, as well as the tenacity of this imbedded particulate in the solidified base coating. When a cross-section of a bundle of filaments is viewed under a microscope, it is apparent that from between about 20 to about 90% of the total surface of each particulate is imbedded into the base coating on the micromesh. This extent of particulate surface imbedding into the base coating is primarily responsible for the “it's working” perception which registers during flossing along with the particulate abrasive retained during handling of the floss prior to flossing (IRF). Permanent abrasives generally exhibit higher IRF values than releasable abrasives.

“Biofilm responsive” is defined as the property of: particulate abrasives, saliva soluble particulates and antimicrobials to work cooperatively with micromesh dental flosses and other cleaning and/or chemotherapeutic substances in the base coating to remove, disrupt and/or control biofilms and the microbiological burden associated with biofilms and residual biofilms while flossing and after flossing with the devices of the present invention.

“Fluidized bed” is defined as a means of converting solid particulate abrasives into an expanded, suspended, solvent-free mass that has many properties of a liquid. This mass of suspended particulate abrasive has zero angle of repose, seeks its own level, while assuming the shape of the containing vessel.

“Sequential fluidized beds” are defined as a means of converting solid particulate abrasives and solid particulate saliva soluble substances separately into expanded, suspended, solvent-free masses that have many properties of a liquid. These separate fluidized masses of suspended particulate abrasive and suspended solid, saliva soluble substances each have zero angle of repose and seek their own level, while assuming the shape of the containing vessel.

“Fibrillating” is generally defined as a means of converting various high tensile strength, stretched film stocks including tapes to various mesh constructions such as illustrated in FIGS. 1a through 1f and shown in photographs in FIGS. 2 through 4 by subjecting the stretched tapes to contact with various rotary fibrillator means such as shown and described in U.S. Pat. Nos. 5,578,373; 2,185,789; 3,214,899; 2,954,587; 3,662,930; 3,693,851 and Japanese Publications: 13116/1961 and 16909/1968. During fibrillating, the transfer speed of the stretched polyethylene tape is from between about 1 and about 1000 m/min and the rotational line speed of the fibrillator means in contact with the stretched polyethylene tape is from between about 10 and about 3000 m/min. These fibrillating conditions produce fibrillated micromesh substrates suitable for various types of coating including compression loading for use as interproximal devices. See FIGS. 1a through 1f and photographs in FIGS. 2 through 4.

“Fibrillation density” is generally defined as the level of perforations in the interproximal device as determined on the basis of the percent of the device surface that is perforated. Perforations between from about 5% and about 90% of the total tape surface area are suitable for purposes of the present invention. There appears to be a correlation between “fibrillation density” and the capacity of the device to entrap and removal loosened substances from interproximal and subgingival areas, i.e., the “entrapment factor”.

“Entrapment factor” is generally defined as the level of loosened biofilm, tartar, debris, food particles, etc., which has been dislodged from tooth surfaces during flossing and subsequently entrapped by the micromesh interproximal device after various coating substances have been released from the “spent” interproximal device. See FIG. 18. The “entrapment factor” is determined by a visual comparison of the spent micromesh interproximal device with a spent commercial monofilament tape used by the same subject at comparable interproximal site. The micromesh interproximal devices of the present invention generally exhibit entrapment factors from between about 2 and about 10 which indicates a two-fold to ten-fold increase in entrapped debris, biofilm, etc., over the commercial monofilament tape.

### BRIEF DESCRIPTION OF THE DRAWINGS

FIG. 1a through 1f are illustrations of uncoated micromesh tapes suitable for the present invention produced by various fibrillations of stretched, ultra-high molecular weight polyethylene tapes.

FIGS. 2a through 2c are actual photographs of uncoated micromesh tapes of the present invention. FIGS. 2d and 2e are photographs of uncoated monofilament dental tape and uncoated micromesh dental tapes, respectively.

FIGS. 3a and 3b are actual photographs of coated micromesh tapes of the present invention where the tapes are at two different levels of fibrillation.

FIGS. 4a through 4c are actual photographs of micromesh tape. FIG. 4a is the tape uncoated. FIGS. 4b and 4c show the tape coated.

FIG. 5 is a schematic side view of a particulate overcoating system of the invention suitable for overcoating saliva soluble, coated, micromesh devices with imbedded, particulate, soft abrasive and imbedded, saliva soluble, solid substances containing flavorants, mouth conditioners, nutraceuticals and/or active therapeutic ingredients.

FIG. 5a is a schematic side view of a particulate overcoating system as shown in FIG. 5, with the filter means replaced by fitted means to recover the particulate overspray that does not contact the substrate during the overcoating operation.

FIG. 6 is an enlarged top view of the system shown in FIG. 5 showing saliva soluble, base coated, micromesh dental floss passing through the particulate coating chamber.

FIG. 7 is an expanded, schematic, three-dimensional view of a coated micromesh dental device showing a saliva soluble, liquid coating on the micromesh dental floss prior to the coated floss entering the particulate coating chamber.

FIG. 8 is an expanded, schematic, three-dimensional view of a saliva soluble, coated, micromesh dental floss showing particulate abrasive imbedded into the liquid base coating after the micromesh dental floss passes through the particulate abrasive coating chamber.

FIG. 9 is an expanded, schematic, three-dimensional view of a base coated, micromesh dental floss showing particulate abrasive partially imbedded into the solidified coating after the particulate abrasive overcoated, micromesh dental floss has been passed through a cooling zone, thereby solidifying the base coating (the cooling zone is not shown).

FIG. 10 is a blown up schematic, partial cross-sectional view of saliva soluble, coated, micromesh dental floss showing particulate abrasive partially imbedded into the solidified base coating which functions as a cushion for the abrasive.

FIG. 11 is a blown up schematic, horizontal, three-dimensional view of a saliva soluble, coated, micromesh dental floss showing a mixture of particulate abrasive and saliva soluble flavor/mouthfeel containing particulates partially imbedded into the solidified base coating.

FIG. 12 is a schematic side view of an alternative particulate overcoating system of the present invention suitable for overcoating saliva soluble, base coated micromesh devices.

FIG. 13 is a schematic side view of another alternative particulate overcoating system of the present invention suitable for overcoating saliva soluble, emulsion coated micromesh devices where the particulate used for overcoating is not detailed.

FIG. 14 is similar to FIG. 9, with the particulate used for overcoating shown in detail.

FIG. 15 is a schematic flow chart for particulate overcoating of saliva soluble, coated, micromesh dental floss.

FIGS. 16 through 18 are schematic illustrations of devices of the present invention being used to remove, disrupt and control biofilms physically and to chemotherapeutically, topically treat residual biofilm remaining after flossing with the substantive, antimicrobial, chlorhexidine digluconate.

### DETAILED DESCRIPTION OF THE PREFERRED EMBODIMENTS

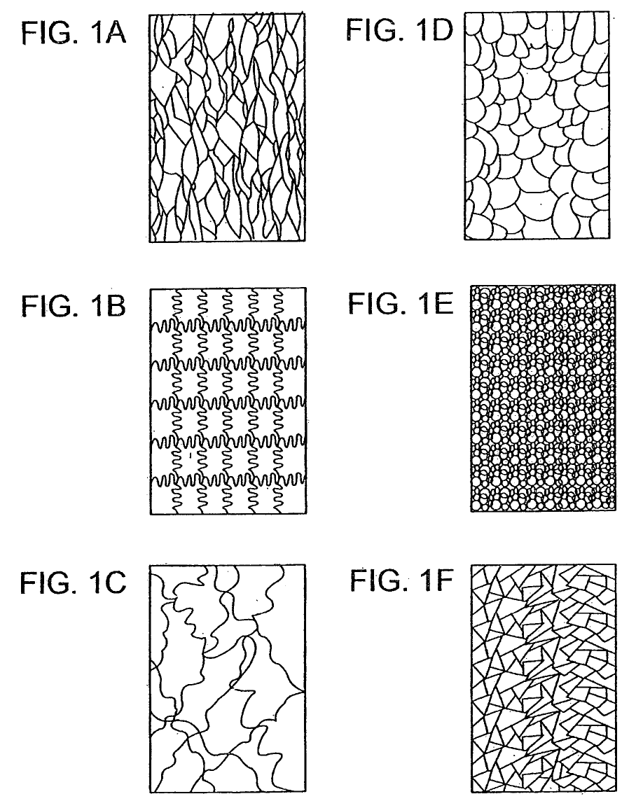

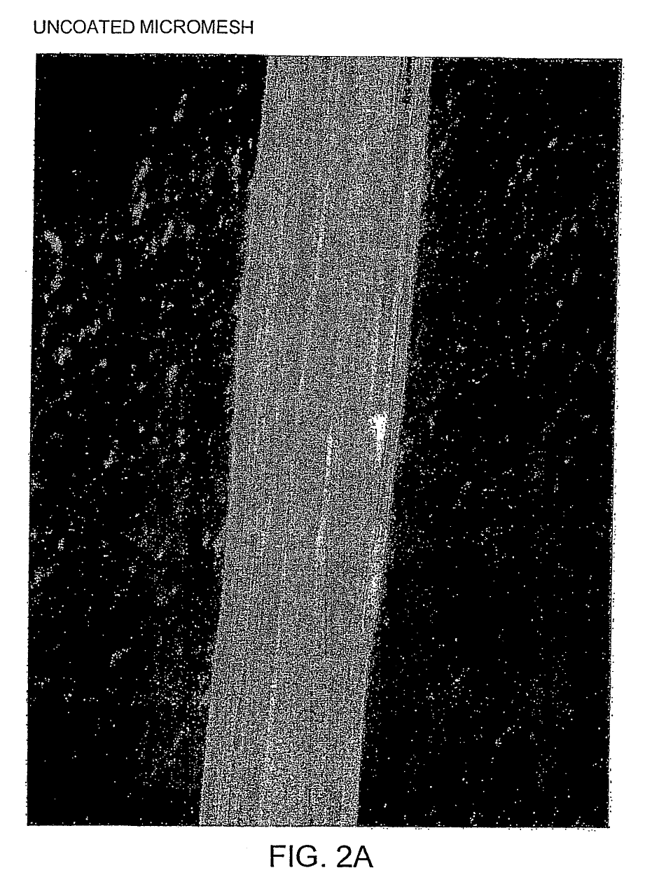

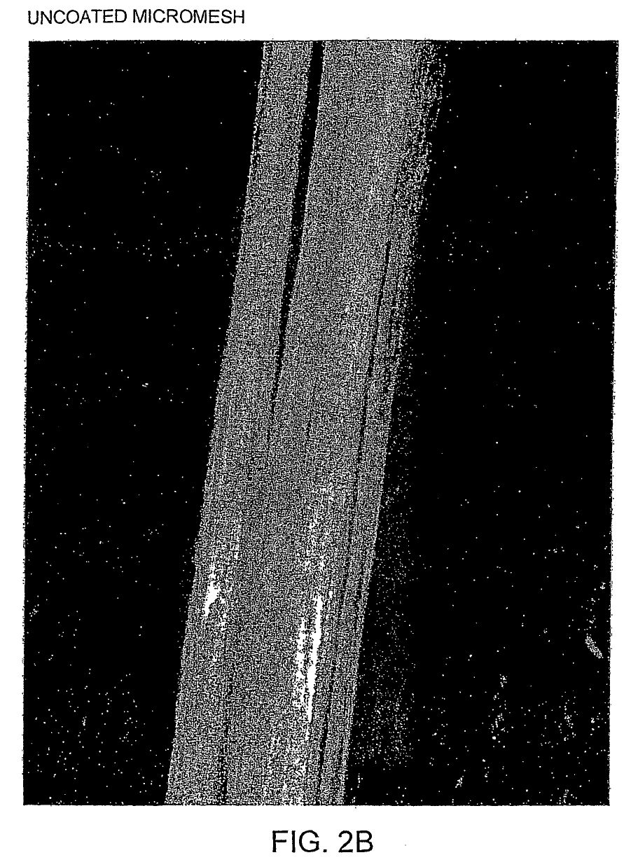

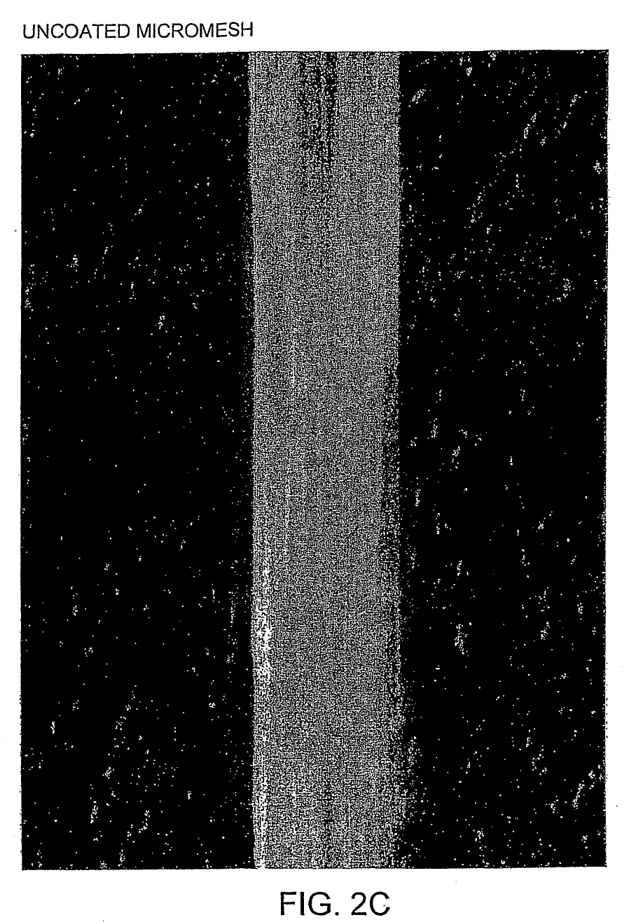

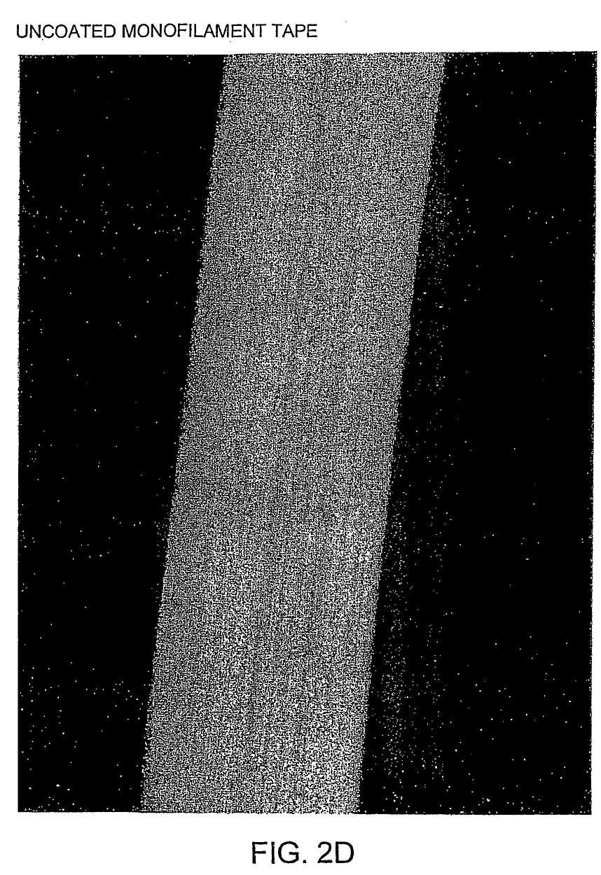

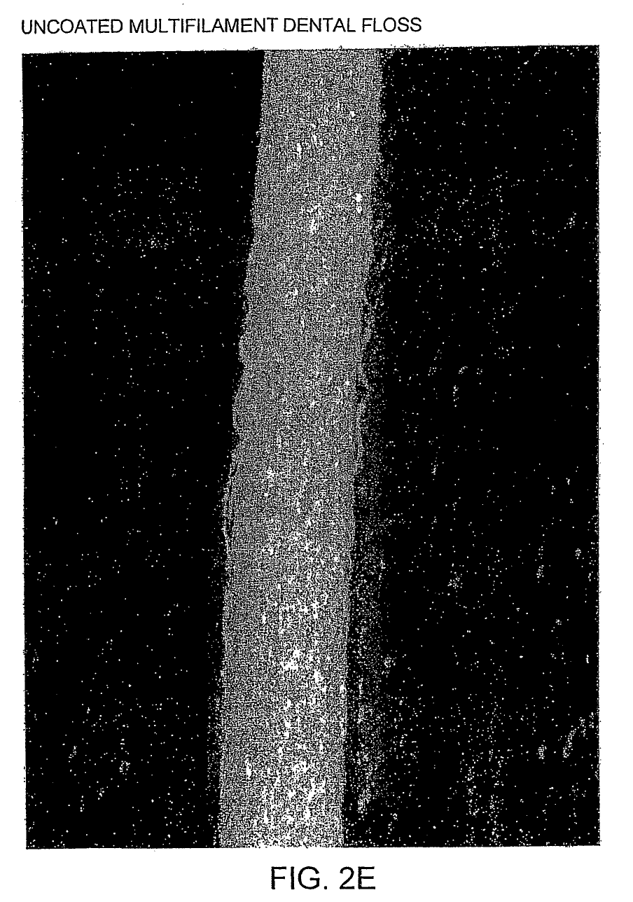

Referring to FIGS. 1 through 4, micromesh devices are distinct from and superior over multifilament dental flosses, as well as monofilament dental tapes. These superior performing interproximal devices are neither multifilament nor monofilament in structure. Rather, they are characterized by a unique micromesh honeycomb or web-type structure, hereinafter described as a micromesh structure shown in FIGS. 1a through 1f. These micromesh devices are not produced from a bundle of fibers like multifilament dental flosses nor are they produced by slitting shred-resistant films used to manufacture PTFE tape or by extrusion used to manufacture elastomeric monofilament tapes and/or the extrusion and slitting processes used to make typical high density polypropylene or polyethylene tapes. Rather, these ultra shred-resistant micromesh devices are produced by fibrillating, meshing, webbing, etc., high-tensile strength, ultra-high molecular weight, stretched, polyethylene films. Generally, this is a penetrating, tearing-type function. This fibrillation of stretched polyethylene films produces various micromesh structures such as illustrated in FIGS. 1a through 1f and further depicted in the photographs in FIGS. 2 through 4.

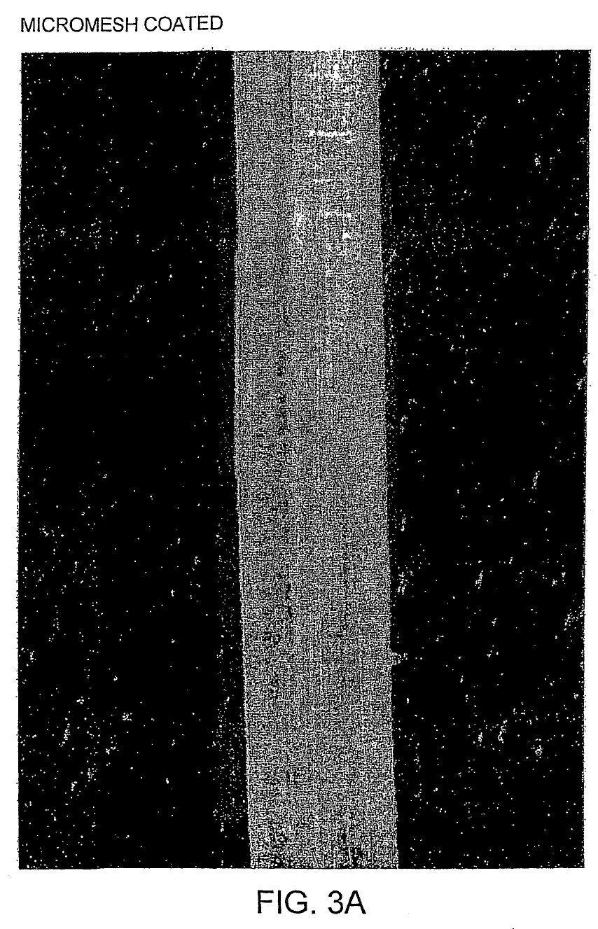

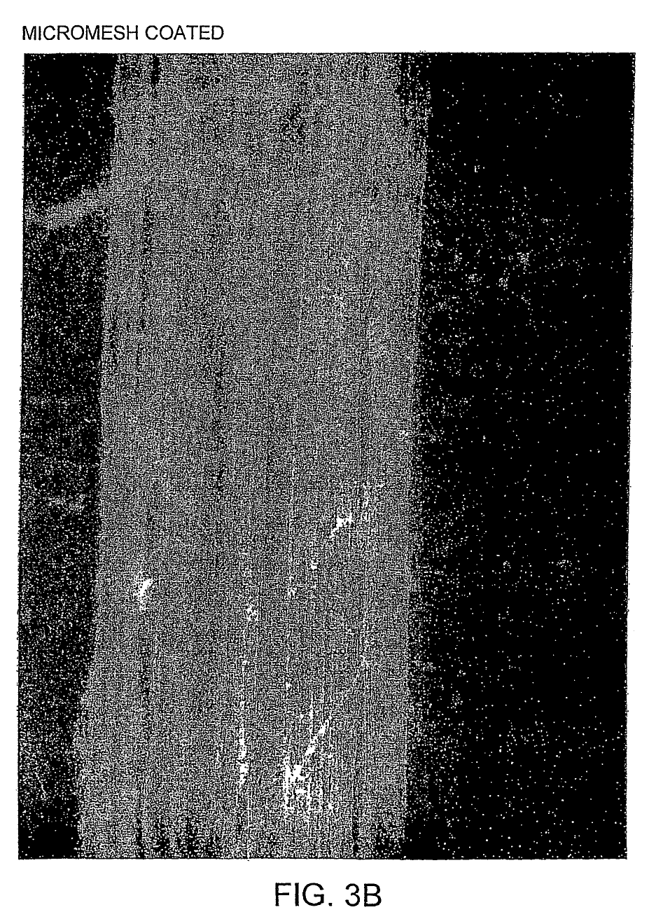

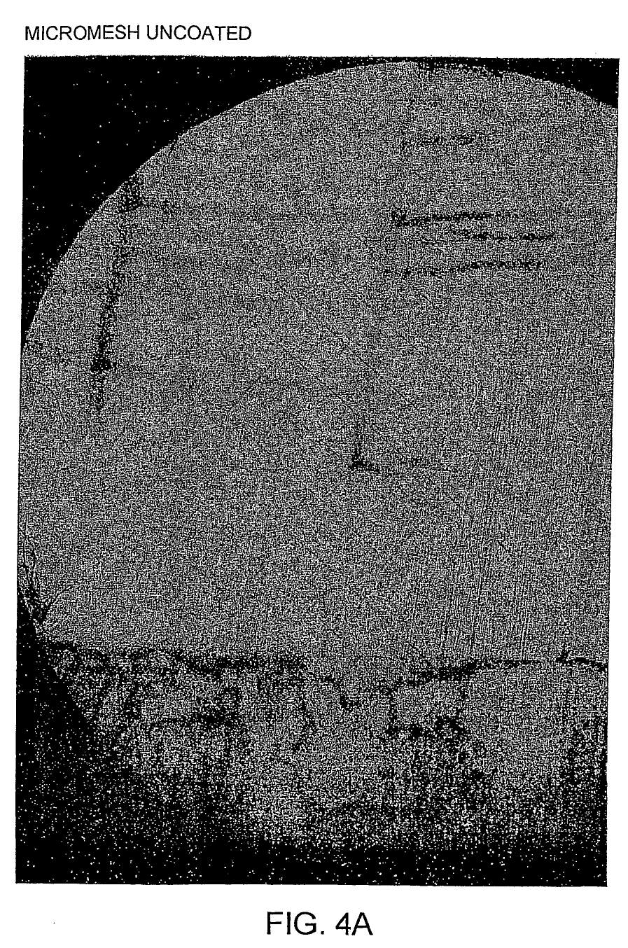

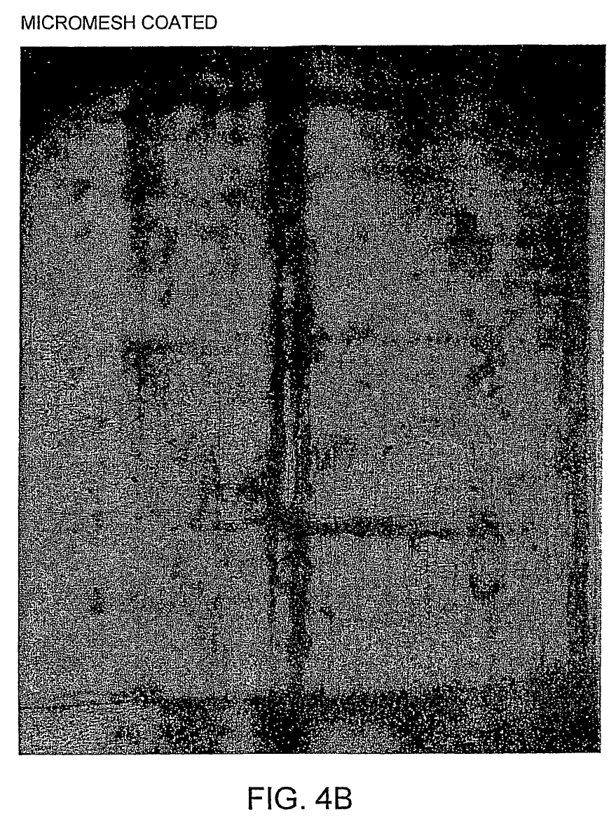

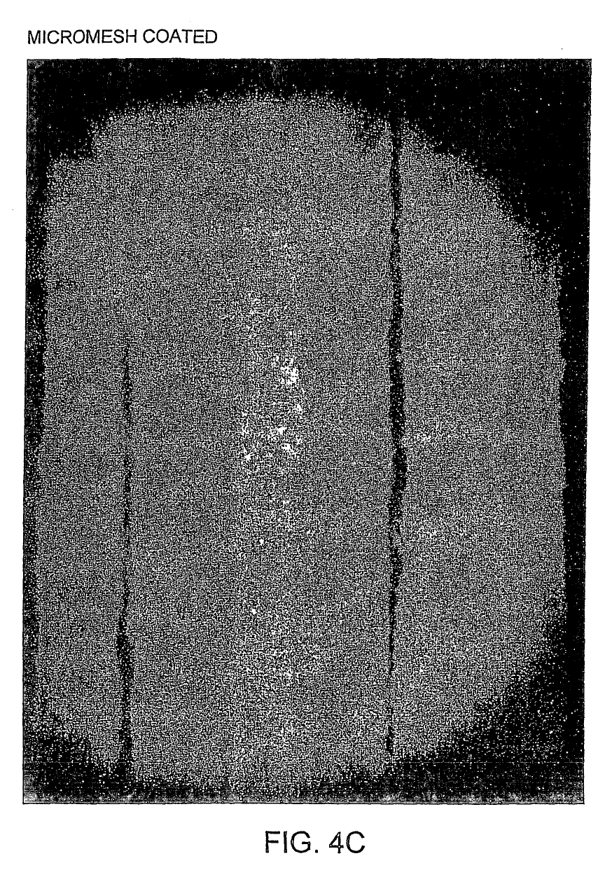

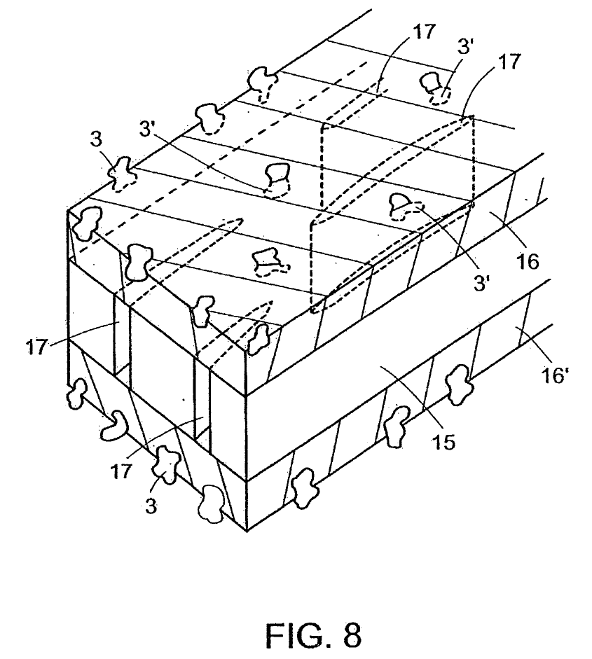

The photographs in FIG. 2 compare typical uncoated multifilament and monofilament devices with uncoated micromesh tapes of the present invention. The photographs in FIG. 3 show coated micromesh interproximal devices at two different levels of fibrillation. The photographs in FIG. 4 illustrate a micromesh tape with a base coat coated and uncoated. Particulate overcoated, coated micromesh flosses of the invention are illustrated in FIGS. 8 through 11.

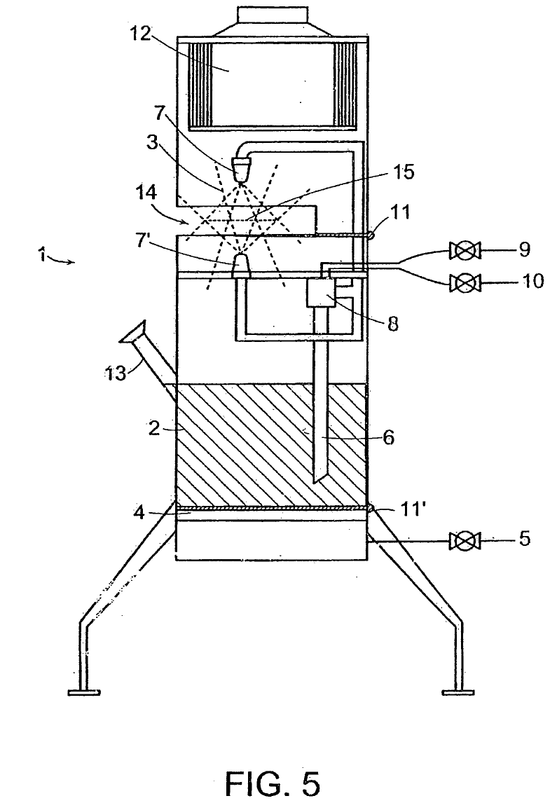

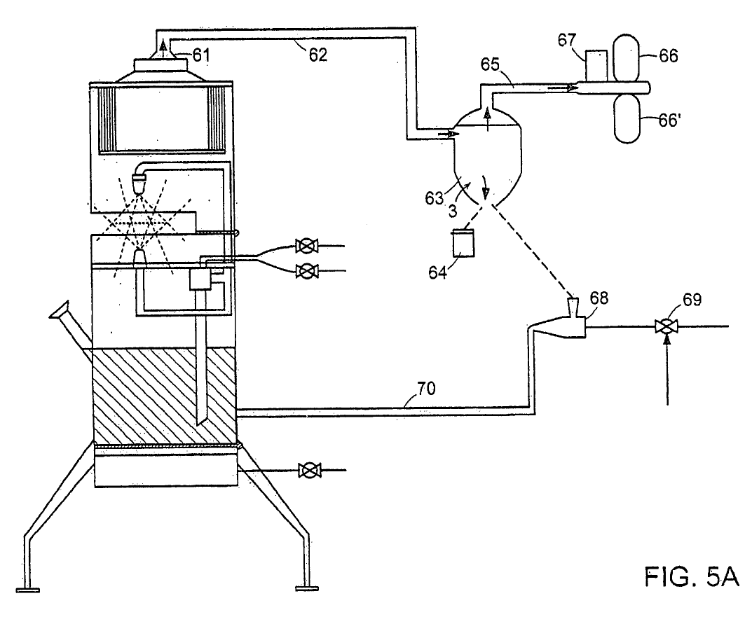

Referring to FIG. 5 which is a schematic side view of a particulate abrasive overcoating system comprising: particulate coating system, 1, consisting of fluidized bed means, 2, comprising: fluidized particulate abrasive, 3, membrane, 4, fluidizing air means, 5, stand pipe, 6, in communication with particulate abrasive nozzle means, 7, provided with pump means, 8, which contains nozzle air input means, 9, and pump cleaning means, 10.

Particulate coating system, 1, is provided with hinged access means, 11 and 15, and filter means, 12, particulate filling means, 13, and coated micromesh dental floss particulate coating zone, 14, and saliva soluble coated micromesh dental flosses, 15. Filter means, 12, can be assisted by a vacuum cyclone means which captures all unused particulate, 3, overspray and recycles same. This is detailed in FIG. 5a.

Saliva soluble, coated micromesh dental floss, 15, with a liquid coating contained thereon, passes through particulate coating zone, 14, where particulate, 3, is imbedded into the liquid coating on micromesh dental floss, 15, from nozzle means, 7.

Referring to FIG. 5a, vacuum cyclone means, 60, replaces former filter means, 12, and is connected to the top of particulate coating system, 1, at juncture 61, via tubing means, 62. Vacuum cyclone means, 60, maintains a slight negative pressure within particulate coating system, 1, by drawing air and some dispersed particulate from coating system, 1, and introducing this air/particulate mixture into vacuum cyclone chamber, 63, where particulate, 3, is introduced into holding means, 64, and the remaining air substantially free from particulate, 3, passes through the top of chamber, 63, through tubing, 65, via motor, 67, into filter means, 66 and 66′. Alternatively, particulate, 3, is captured by collecting means, 68, with air regulator, 69, and returned to particulate coating system, 1, via tubing, 70.

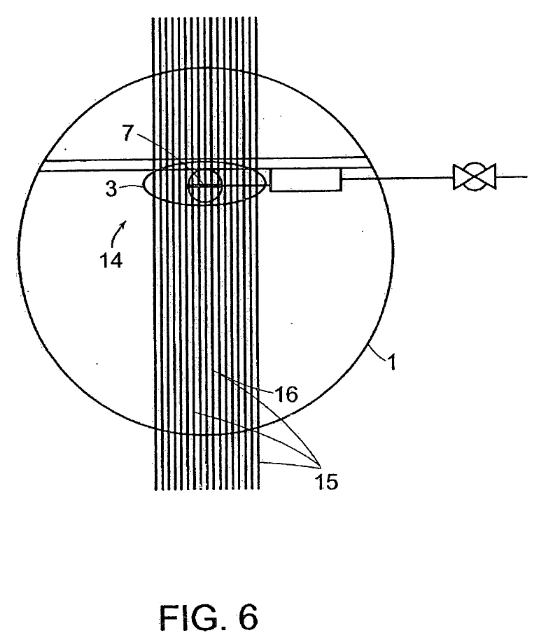

Referring to FIG. 6, which is an enlarged top view of particulate coating system, 1, shown in FIG. 5. Micromesh dental floss, 15, with saliva soluble liquid base coating, 16, thereon, passes through particulate coating zone, 14, where particulate abrasive, 3, from nozzle means, 7, is imbedded via impinging into liquid base coating, 16, which is substantive to the micromesh dental floss, 15, as micromesh dental floss, 15, passes through particulate coating zone, 14.

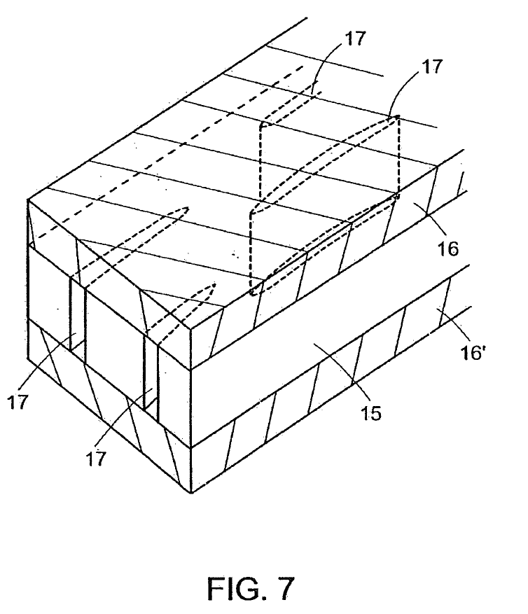

Referring to FIG. 7, which is an expanded, schematic, three-dimensional view of coated micromesh dental floss, 15, with fibrillations, 17, showing base saliva soluble liquid coating, 16, thereon before the floss, 15, passes into particulate coating zone, 14. The saliva soluble coating, 16, has been heated and is in a liquid state and is substantive to the micromesh floss web, 15.

Referring to FIG. 8, which illustrates an expanded, schematic, three-dimensional view of saliva soluble emulsion coated micromesh floss, 15, with fibrillations, 17, showing saliva soluble base coating, 16, containing particulate abrasives, 3, imbedded into the liquid coating, 16, with the imbedded portion of the particulate abrasive shown via dotted lines designated as 3′.

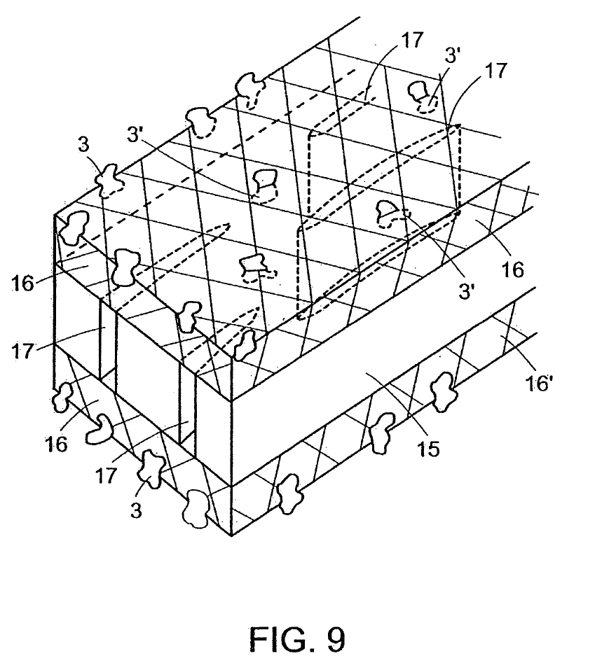

Referring to FIG. 9, which is an expanded, schematic, three-dimensional view of saliva soluble emulsion coated micromesh dental floss, 15, with fibrillations, 17, showing saliva soluble base coating, 16, that has been passed through a cooling zone (not shown) sufficient to solidify said base coating, 16, with particulate abrasive, 3, firmly imbedded into said solidified base coating, 16, with the imbedded portion of the particulate abrasive represented by the dotted lines designated as 3′.

Referring to FIGS. 5 and 9, in a particularly preferred embodiment of the invention, the particulate overcoating system, 1, set forth in FIG. 5, is replicated and in line, in order to sequentially imbed two distinct particulate substances having substantially different densities onto the saliva soluble base coating, 16, on micromesh, 15. Under this sequential particulate coating operation, particulate substance abrasive, 3, imbeds into saliva soluble coating, 16, prior to the particulate overcoated floss, 15, passing directly from a first particulate coating zone, 14, into a second similar particulate coating zone, where a high impact particulate mouth conditioning substance is also imbedded into base coating, 16, prior to the multi-particulate overcoated floss, 16, passing to the cooling zone, not shown. In this sequential arrangement, two distinct particulates having substantially dissimilar densities are imbedded into the saliva soluble liquid base coating, 16, using this sequential fluidized bed arrangement prior to said saliva soluble base coating solidifying.

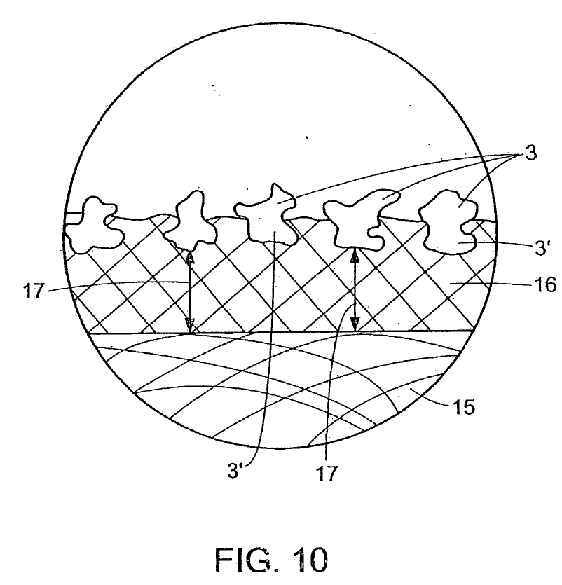

Referring to FIG. 10, which is an expanded, schematic, partial cross-sectional view of saliva soluble emulsion coated micromesh dental floss, 15, showing solidified base coating, 16, with particulate abrasive, 3, firmly partially imbedded in solidified saliva soluble base coating, 16, with “cushion”, 19, extending from the bottom of particulates, 3, to the surface of micromesh dental floss, 15. The imbedded portion of the particulate abrasive is designated as 3′.

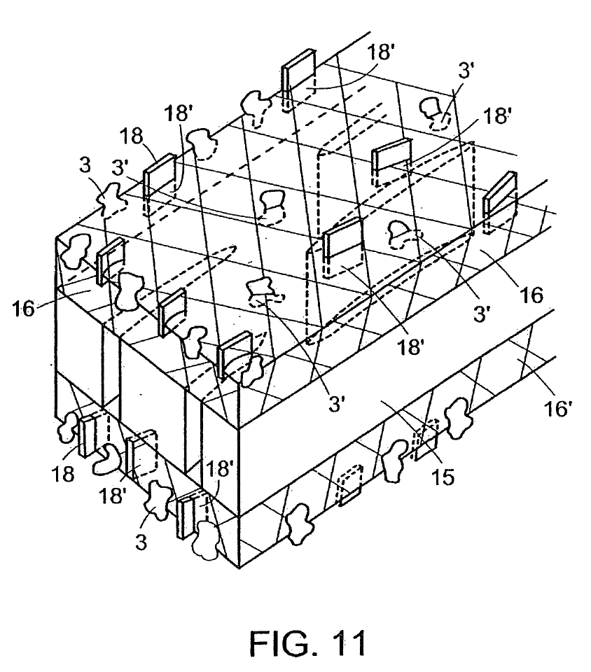

Referring to FIG. 11, which is an expanded, schematic, horizontal, three-dimensional view of emulsion coated micromesh dental floss, 15, showing a mixture of particulate abrasive, 3, and saliva soluble particulate, mouth feel, mouth conditioning, substance, 18, each shown firmly partially imbedded into said solidified saliva soluble base coating, 16, with the imbedded portions of 3 and 18 shown by dotted lines, 3′ and 18′, respectively.

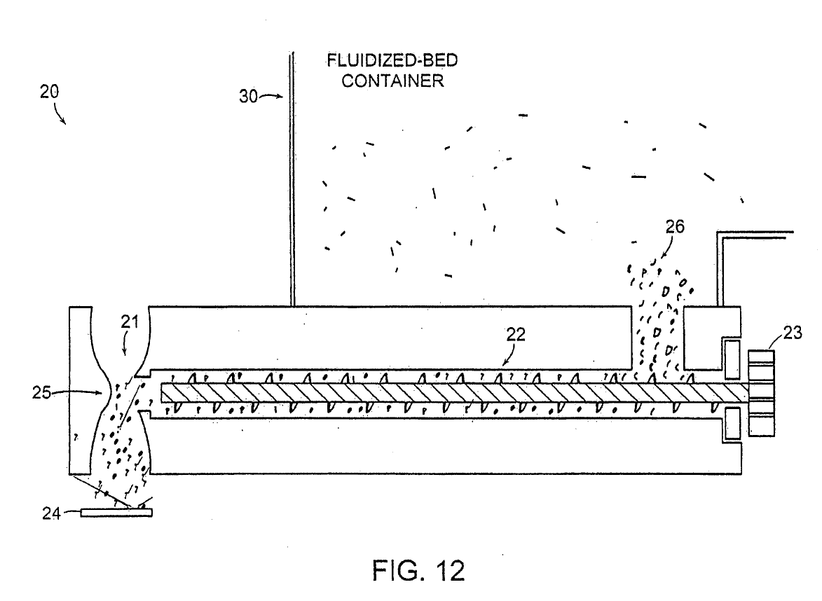

Referring to FIG. 12, which is a schematic side view of an alternative particulate overcoating system, 20, for delivering a particulate, 21, from a vessel or fluidized-bed means, 30, to a conveying agent means, 22, with gear drive means, 23. The speed of conveying auger, 22, is controlled by motor driven gear means, 23, which is slaved to a surface speed controller, not shown, for micromesh floss, 24. As the micromesh floss, 24, moves faster, auger means, 22, speeds up and delivers more particulate, 21, to the surface of molten-coated micromesh floss, 24. This system then allows for the delivery of a constant density of particulate, 21, per square millimeter of micromesh floss, 24. This alternative particulate overcoating system requires substantially lower volumes of air with corresponding reductions in overspray of particulates. This system requires minimal recovery of unused particulate and/or recycling of unused particulates.

In the foregoing system, the particulate, 21, may be an abrasive such as pumice, having an average particulate size of 37 microns which are fluidized with a porous plate of sintered polyethylene powder of 0.5 inch thickness. The plate has an average pore size of 20 microns. As the fluidized pumice is presented to auger means, 23, it is pulled down the shaft and presented to venturi means, 25. Control of the air flow in proportion to the speed allows uniform delivery of pumice to a surface of micromesh floss, 24, passing under the outlet of venturi means, 25. This arrangement allows delivery of uniform particle density with very low air speed, consistent with little perturbation of the floss traverse.

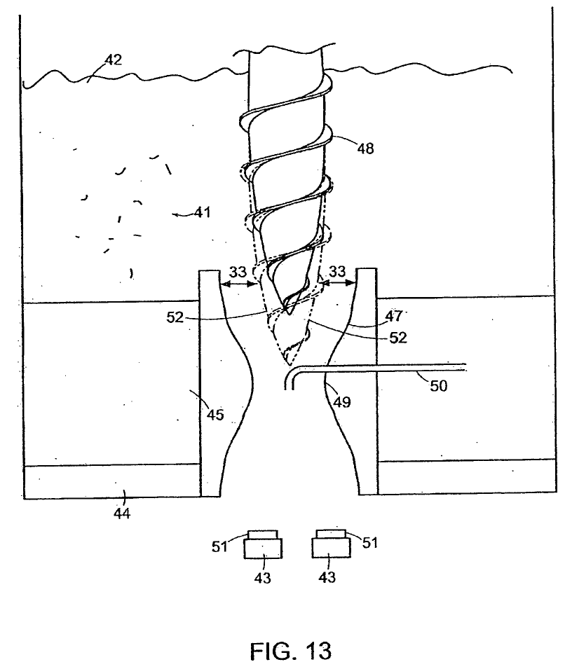

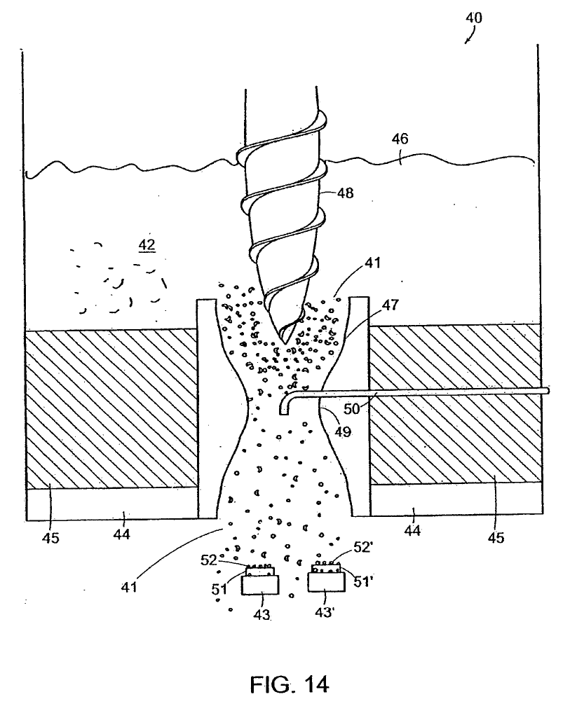

Referring to FIGS. 13 and 14, which are two separate schematic side views of another alternative particulate overcoating system, 40, for delivering particulates, 41, from a fluidized bed means, 42, to micromesh flosses, 43 and 43′.

Air chamber means, 44, introduces air under low pressure through distributor plate means, 45, which in turn fluidizes particulates, 41, in fluidized bed means, 46. Particulates, 41, are introduced from fluidized bed, 46, into particulate coating chamber, 47, by particulate metering means, 48. Particulate coating chamber, 47, is provided with venturi means, 49. Modulating particulate dispensing means, 50, is provided with high velocity, low volume air means (not shown) providing turbulence to fluidized particulate, 41, prior to said particulate imbedding coatings, 51 and 51′, on the micromesh web, 43 and 43′, respectively. Particulate dispensing means, 50, enhances the uniformity of the particulate, 41, overcoating, 52 and 52′, imbedded into coatings, 51 and 51′, respectively.

Referring to FIG. 13, generally the pressure in air chamber, 44, is between 4 and 8 psi. Distributor plate, 45, is preferably a porous polyethylene means that creates air bubbles required to fluidize particulates, 41, in fluidized bed, 42. The air pressure in fluidized bed, 42, is preferably in the 0.2 to 0.5 psi range. Particulate metering means, 48, can take many shapes other than that of the threaded means depicted. For example, metering means can be a plug or ram without threads that controls the flow of particulates, 41, from fluidized bed, 42, into particulate coating chamber, 47. Lowering metering means, 48, into particulate coating chamber, 47, as shown by dotted lines, 52, further restricts the flow of fluidized particulate, 41, through distance, 53. Thus, particulate metering means, 48, determines the quantity of fluidized particulate, 41, to enter particulate metering area, 47. This control in combination with modulated air flow through particulate dispersing means, 50, produces a substantially uniform density particulate on saliva soluble coating, 51, with imbedded particulates, 52, being dispersed substantially uniformly throughout saliva soluble coating, 51.

For a production system comprising up to 32 micromesh lines running side-by-side, the particulate overcoating system, 40, will be replicated in groups of 8, with two such groups covering the total of 32 lines running side-by-side.

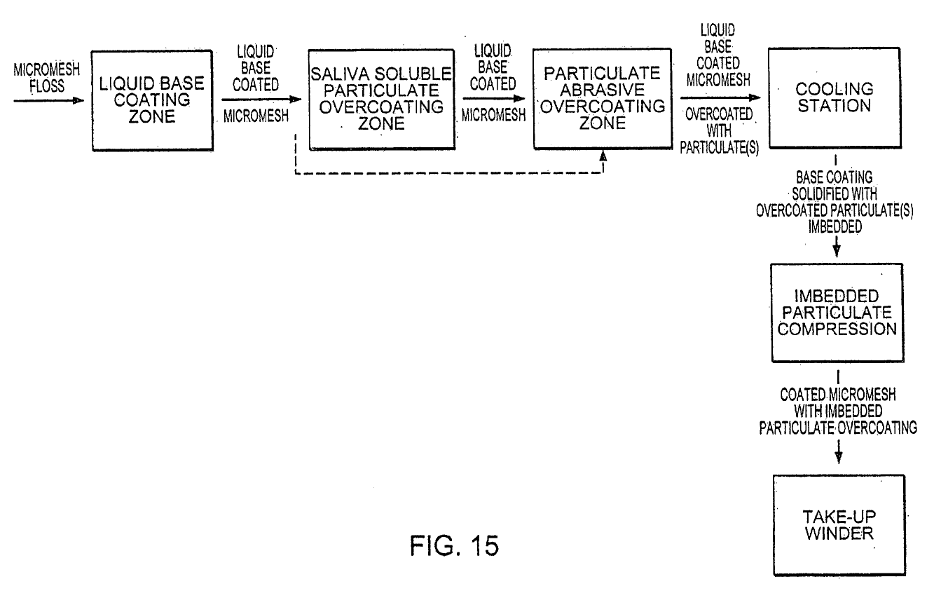

Referring to FIG. 15, which is a schematic flow chart for particulate overcoating of saliva soluble coated micromesh dental floss, micromesh floss is passed through liquid base coating zone where the saliva soluble base coating containing an antimicrobial is applied. Particulate overcoating is applied by introducing the coated micromesh into one or two particulate overcoating zones, after which the particulate overcoated micromesh floss passes through a cooling zone, followed by passing the overcoated micromesh through a particulate compression means before being introduced to a take-up winder means.

Referring to FIGS. 16 through 18, the following mechanism of action is illustrated:

A. During Flossing:

Step 1: The saliva soluble base coat at 80 mg/yd containing 3.8 mg/yd chlorhexidine digluconate is released from the fibrillated tape, along with the overcoating of 4 mg/yd SOFT ABRASIVES®.
Step 2: The released saliva soluble coating proceeds to clean and coat teeth and soft tissue surfaces before dissipating into the saliva flow.
Step 3: The insoluble SOFT ABRASIVES® particulate overcoating, which has released from the substrate, prior to being flushed away by the saliva, is worked over interproximal tooth surfaces, subgingivally and supragingivally, by the fibrillated substrate that is now substantially free of the saliva soluble coating.
This SOFT ABRASIVES®/fibrillated substrate combination removes, disrupts and controls biofilm and chlorhexidine-stained pellicle until such time as the SOFT ABRASIVES® are flushed away by the saliva and the tape is removed.
Step 4: The released substantive chlorhexidine digluconate attaches to residual biofilm not removed by flossing, as well as to the pellicle present on tooth surfaces.
   B. After Flossing:
Step 1: The spent fibrillated substrate, which is removed from interproximal spaces, contains entrapped:
   loosened biofilm,
   loosened stained pellicle,
   stained microbiota
   food particles,
   debris,
   materia alba, etc.
Step 2: Chlorhexidine antimicrobial remains substantive for up to 8 hours, disrupting and controlling the microflora in the residual biofilm and resisting being flushed away by the saliva.
Step 3: The mouth “feels and tastes” fresh and clean with no perceived chlorhexidine tooth staining and/or aftertaste.

The micromesh floss devices of the present invention can contain a broad range of saliva soluble coating substances which are best loaded onto and/or into the micromesh structure by one of three loading means. Specifically:
1. The high melt viscosity mixtures and emulsions are loaded onto and/or into the micromesh by compression means;
2. The medium melt viscosity mixtures and emulsions are loaded onto and/or into the micromesh by injection loading means; and
3. The low melt viscosity mixtures and emulsions are loaded onto and/or into the micromesh by contact loading means.

The improved interproximal devices of the present invention contain base coatings that: (a) comprise from 10 to 120% by weight of the micromesh substrate, (b) are preferably saliva soluble and (c) in a preferred embodiment are crystal free, and accordingly, exhibit a minimum of flaking. Some of these base coatings are released in total into the oral cavity during flossing.

In a preferred embodiment, these base coatings contain ingredients such as: (a) antimicrobials such as chlorhexidine digluconate, (b) SOFT ABRASIVES® that work with the micromesh structure to help physically remove biofilm (plaque) from interproximal, supragingival and subgingival surfaces, (c) other chemotherapeutic ingredients affecting oral health and subsequent systemic diseases caused or exacerbated by poor oral health, (d) cleaners that introduce detersive effects into the areas flossed, and (e) mouth conditioners. These base coatings are particularly adapted to loading into and/or onto the micromesh tapes using the compression, injection or contact loading means described above to produce the innovative interproximal devices of the present invention.

The particulate abrasives and other saliva soluble particulate substances of the present invention are overcoated into the coated micromesh dental floss base coatings as solid materials substantially free from solvents.

A preferred method of imbedding particulate abrasive overcoatings and saliva soluble particulate overcoatings into the base coat of the micromesh device is by means of a series of innovative fluidized bed systems such as the system shown in FIG. 5.

Referring to FIG. 5, membrane means, 4, is used to maintain the particulate abrasive, 3, or saliva soluble particulate, 18, in a state of continued fluidization, i.e., fluidized bed, 2. Particulate abrasive, 3, or saliva soluble particulate, 18, can each be maintained in a fluidized state using fluidizing bed, 2. These fluidized particulates are introduced essentially at a 90° angle to the traverse of coated micromesh dental floss, 15, via nozzle means, 7 and 7′, through stand pipe means, 6, via pump means, 8.

Referring to FIG. 5, saliva soluble, coated, micromesh dental floss, containing an antimicrobial, 15, passes through particulate coating zone, 14, and is imbedded with particulate abrasive, 3, as shown in FIGS. 8 thru 10, or with saliva soluble particulate, 18, as shown in FIG. 11. Particulate abrasive, 3, and saliva soluble particulate, 18, are each separately introduced under high impact conditions into liquid base coating, 16, on micromesh floss, 15, via nozzle means, 7 and 7′, via separate particulate overcoating system positioned sequentially in a series immediately prior to the particulate overcoated micromesh flosses entering the cooling zone, not shown.

Imbedding of the particulate abrasive, 3, into the saliva soluble base coating, 16, throughout the coating on the micromesh, 15, is achieved by means of impinging said particulate into the hot, liquid, base coating that is present over the entire outer surface of said micromesh device at the time the particulate abrasive, 3, impinges the coating, 16. See FIGS. 8 thru 10.

That is, the particulate abrasive, 3, impinges into liquid saliva soluble coating, 16, which is substantive to micromesh web, 15, as the device passes through particulate coating zone, 14, and particulate abrasive, 3, is imbedded into coating, 16, as shown in FIG. 9 and in solidified coating, 16, as shown in FIGS. 10 and 11.

That is, particulate abrasive, 3, impinges into the hot, viscous, saliva soluble, base coating containing an antimicrobial, 16, which is a viscous liquid generally at a temperature between about 48° C. and 110° C. with a viscosity between 10 and 10,000 cs. This is illustrated in FIGS. 8 and 9, with the exposed portion of particulate abrasive designated as 3, and the imbedded portion of the particulate abrasive indicated by dotted lines and designated as 3′.

The micromesh dental floss overcoated with imbedded particulate then proceeds through a cooling means (not shown), where the base coating, 16, cools and solidifies with the particulate abrasive, 3, and the antimicrobial imbedded therein, as illustrated in FIGS. 9 through 11.

FIG. 11 illustrates high-impact particulate overcoating into a micromesh dental floss base coating. That is, the particulate abrasive, 3, and particulate saliva soluble substances, 18, that contain mouth conditioners, flavorants, active ingredients, etc. are imbedded into the base coating, 16, as illustrated in FIG. 11. Particulate abrasive, 3, along with saliva soluble particulate substance, 18, are sequentially imbedded into base coating, 16, on micromesh floss, 15, from separate fluidized bed sources prior to base coating, 16, solidifying.

The overcoatings of particulate abrasive and various saliva soluble particulate substances containing flavorants and/or mouth conditioners and/or chemotherapeutic substances can include a broad range of these substances. For example, particulate ratios of particulate abrasives to saliva soluble substances such as nonionic surfactants (PLURONICS®), emulsions such as MICRODENT® and/or ULTRAMULSIONS® and/or polyols such as PEG in these hi-impact particulate overcoatings can range from 10:90 to 90:10.

The innovative fluidized bed coating process of the present invention is most effective in imbedding:
(1) particulate abrasive loads between about 2 and about 45 percent by weight into the saliva soluble, coated device containing an antimicrobial,
(2) particulate, saliva soluble loads between about 2 and about 45% by weight into the coated device,
(3) particulate abrasive overcoating into coated micromesh devices with a perceived abrasive factor (PAF) between about 2 and 4, and
(4) particulate abrasive, overcoating into coated micromesh devices with an Incidental Release Factor (IRF) value well above 80%, and preferably over 90%, and most preferably over 95%.

It has been discovered that in order to produce a coated micromesh dental device with PAF values in the 3 to 4 range, it is necessary: (1) to embed particulate abrasive loads at between about 10 and 34 percent by weight of the device, (2) to restrict the average particle size of the imbedded particulate abrasive to between about 7 microns and about 200 microns, (3) to restrict the particle size distributions of the imbedded particulate abrasive to from between about 5 microns and about 300 microns, and (4) to imbed the particulate abrasive into the saliva soluble liquid base coating under a high velocity charge from several nozzle means positioned at 90° to the traverse of the coated micromesh floss through the particulate coating chamber, thereby maximizing the impingement of the particulate abrasive into the base coating.

Overcoating coated micromesh floss with saliva soluble particulate can be carried out by imparting a static charge to the saliva soluble particulate prior to discharge from the nozzle means. Means are provided for grounding the liquid, base, coated micromesh in order to receive the charged saliva soluble particulate. Alternatively, saliva soluble particulate can be imbedded into liquid base coatings on micromesh dental flosses by various spraying means.

In addition to various types of fluidized bed/nozzle arrangements, the particulate abrasive overcoatings can be imbedded into the coated micromesh dental flosses by several other means for impinging particulate abrasives onto liquid coated micromesh. These include various powder coating processes including fluidized bed, plastic frame-spraying, electrostatic spraying and sonic spraying. In the latter, sound waves are used to suspend the particulate abrasives before introducing the fluidized particulate abrasive into a nozzle means.

Other particulate abrasive overcoating processes are described in U.S. Pat. Nos. 6,037,019; 3,848,363; 3,892,908; 4,024,295; 4,612,242; 5,163,975; 5,232,775; 5,273,782; 55,389,434; 5,658,510; 2,640,002; 3,093,501; 2,689,808; 2,640,001 and 5,194,297. These can be adapted to particulate abrasive impingement on coated micromesh as taught by the present invention and are incorporated herein by reference.

Particularly preferred particulate overcoating means include various Nordson® automatic powder coating systems such as the Nordson® Tribomatic II powder coating system, which includes various Nordson® powder pumps, as well as ITW Gema Powder coating systems including their Easysystem™ and Electrostatic Equipment Co's 7R FLEXICOAT® system.

The particulate overcoating of the invention can be affected with various other means for delivering particulate to the saliva soluble liquid base coating. For example, the particulate can be introduced by a simple screening technique where the particulate drops from the screening means onto the liquid means onto the liquid base-coated micromesh.

The preferred means of the invention for overcoating includes a fluidized bed in combination with a nozzle means. This combination provides the most uniform overcoatings while controlling the extend of the particulate imbedding into the liquid base coating and optimizing PAF and IRF values.

Various dental particulate abrasives imbedded into a standard saliva soluble, coated, micromesh dental floss containing an antimicrobial having an average denier of 840 and a base coating of about 25 mg/yd, suitable for purposes of the present invention, are illustrated in Examples 1 through 7, as described in detail in Table 1 below:

TABLE 1
“Dental” Particulate Abrasives suitable for imbedding into coated micromesh dental flosses

| | | | | Particulate | Projected | Projected | |
|---|---|---|---|---|---|---|---|
| | | | Particle Size | Abrasive Load | Incidental | Perceived | Estimated % of total particulate |
| | Particulate | Avg. Particle Size | Distribution | as % by wt. of | Release Factor | Abrasive Factor | abrasive surface area imbedded |
| Example # | Abrasive(s) | (in microns) | (in microns) | device | (IRF) in % | (PAF) | into coated micromesh floss |
| 1 | pumice | 35 | 4-120 | 23 | 95 | 3.5 | 14 to 19 |
| 2 | silica | 10 | 2-18 | 10 | 98 | 1.5 | 6 to 9 |
| 3 | pumice & silica | 12 | 2-120 | 16 | 96 | 2.5 | 13 to 15 |
| 4 | dicalcium | 55 | 18-100 | 15 | 98 | 1.5 | 12 to 14 |
| | phosphate | | | | | | |
| | dihydrate | | | | | | |
| 5 | alumina | 25 | 10-75 | 20 | 94 | 3.7 | 15 to 18 |
| 6 | calcium carbonate | 50 | 15-80 | 16 | 97 | 2.0 | 13 to 15 |
| 7 | polyethylene | 20 | 8-40 | 12 | 98 | 1.5 | 9 to 11 |

Various “active” particulate abrasives imbedded into a standard coated micromesh dental floss having a denier of 840 and containing about 30 mg/yd base coating, suitable for purposes of the present invention, are illustrated in Examples 8 through 12 as described in detail in Table 2 below:

TABLE 2
“Active” Particulate Abrasives suitable for imbedding into saliva soluble, coated,
micromesh dental flosses containing an antimicrobial

| | | | | Particulate | Projected | Projected | |
|---|---|---|---|---|---|---|---|
| | Active | Avg. | Particle Size | Abrasive Load | Incidental | Perceived | Estimated % of total particulate |
| | Particulate | Particle Size | Distribution | as % by wt. of | Release Factor | Abrasive Factor | abrasive surface area imbedded |
| Example # | Abrasive(s) | (in microns) | (in microns) | device | (IRF) in % | (PAF) | into coated micromesh floss |
| 8 | tricalcium | 60 | 10-150 | 10 | 90 | 3.0 | 7 to 9 |
| | phosphate & silica | | | | | | |
| 9 | tetrapotassium | 65 | 20-175 | 12 | 90 | 2.5 | 8 to 11 |
| | pyrophosphate & | | | | | | |
| | pumice | | | | | | |
| 10 | tetra sodium | 70 | 20-150 | 8 | 90 | 2.5 | 5 to 7 |
| | pyrophosphate | | | | | | |
| 11 | sodium | 75 | 20-175 | 17 | 85 | 3.0 | 12 to 15 |
| | hexametaphosphate | | | | | | |
| | & pumice | | | | | | |
| 12 | calcium | 9 | 4-35 | 20 | 98 | 2.0 | 15 to 19 |
| | pyrophosphate & | | | | | | |
| | silica | | | | | | |

Suitable particulate abrasives for the present invention can also contain active ingredients “dusted” thereon. For example, antimicrobials such as cetylpyridinium chloride, triclosan, chlorhexidine, etc., can be dusted onto the particulate abrasives prior to overcoating the coated micromesh floss. During flossing, these antimicrobial coatings on the particulate abrasives are released therefrom during flossing and remain available interproximally and subgingivally to work with the particulate abrasive imbedded micromesh dental floss during flossing as biofilms are being removed, disrupted and/or controlled.

Suitable emulsion, saliva soluble and flake-free base coatings for various micromesh dental flosses are described in Examples 13 through 27 in Table 3 below:

TABLE 3
Suitable Saliva Soluble Base Coatings for Micromesh Dental Flosses to be overcoated with particulate abrasive

| | EXAMPLE NO. | | | | | | | | | | | | | | |
|---|---|---|---|---|---|---|---|---|---|---|---|---|---|---|---|
| Ingredients | 13 | 14 | 15 | 16 | 17 | 18 | 19 | 20 | 21 | 22 | 23 | 24 | 25 | 26 | 27 |
| Ultramulsion 10/2.5 | | | 57.4 | | 52.1 | 49.4 | 56.9 | | 64.8 | 45.4 | | | | 77.1 | 78.6 |
| Microwax 445 | | | 7.0 | | 7.0 | 7.0 | 7.2 | | | 7.0 | | | | | |
| PEG 40 Sorbitan diiso. | | | 3.0 | | 3.0 | 3.0 | 3.0 | | | 3.0 | | | | | |
| Stearyl alcohol | | | 15 | | 15 | 15 | 15 | | | 15 | | | | | |
| Insoluble saccharin | 2.3 | 1.6 | 1.3 | 1.0 | 2.1 | 1.8 | 1.8 | 2.3 | 2.3 | 1.8 | 2.3 | 2.3 | 2.3 | 2.1 | 2.3 |
| Propyl Gallate | 0.1 | 0.1 | 0.1 | 0.1 | | 0.1 | 0.1 | 0.1 | 0.1 | 0.1 | 0.1 | 0.1 | 0.1 | | 0.1 |
| Flavor | 9.6 | 10.0 | 10.0 | 7.5 | 5.4 | 8.5 | 10.0 | 8.8 | 8.6 | 10.0 | 4.0 | 4.0 | 8.0 | 6.0 | 6.0 |
| Dicalcium dihydrate | | | 6.0 | | | | 3.0 | 13.3 | | | 15.0 | | | | 13.0 |
| phosphate | | | | | | | | | | | | | | | |
| Pumice | | | | | | | 3.0 | | | | | | | | |
| EDTA | | 0.2 | 0.2 | 0.2 | | 0.2 | 0.2 | 0.2 | | | 0.2 | | | | |
| TSPP | | | | | | | | | 13.2 | 6.0 | 4.0 | 26.6 | | | |
| Silica | | | | | 5.0 | 10.0 | | | 4.0 | | | 4.0 | 4.0 | 10.0 | |
| Calcium Peroxide | | | | | | 5.0 | | | | | | | | | |
| Chlorhexidine digluconate | | | | | 4.4 | | | | | | 3.2 | | | | |
| Poloxamer 407 | 53.0 | 35.0 | | 20.0 | | | | 44.4 | | | 61.2 | 45.0 | 19.4 | | |
| PEG 8000 | | | | | | | | | | 11.7 | | | 33.0 | | |
| PEG 1450 | 35.0 | 53.0 | | 71.1 | | | | | 7.6 | | 10.0 | 8.0 | 33.0 | | |
| Sodium fluoride | | 0.1 | | 0.1 | | | | | | | | | 0.2 | | |
| Carrageenin | | | | | | | | 13.3 | | | | | | | |
| Silicone (PDMS) | | | | | | | | 17.6 | | | | 10.0 | | | |
| SnF₂ | | | | | | | | | | | | | | 4.8 | |

### EXAMPLE 28

A saliva soluble base coating containing chlorhexidine digluconate for micromesh dental floss was prepared having the following formula:

| Ingredient | Grams |
|---|---|
| Ultramulsion 10/2.5 | 473 |
| Stearyl alcohol | 150 |
| Emsorb 2726 | 30 |
| Propyl Gallate | 1 |
| Mult wax ML-445 | 70 |
| Insoluble saccharin | 18 |
| Sident 10 | 100 |
| Peppermint flavor | 100 |
| Chlorhexidine | 56 |
| digluconate | |
| EDTA | 2 |
| Total | 1000 |

The foregoing was added to micromesh dental floss at various rates. This coated micromesh can be overcoated with various particulate abrasives at various rates, as detailed in Table 4 below.

TABLE 4
Coated Micromesh Chlorhexidine Dental Floss, Particulate Abrasive Overcoating Data

| | Micromesh | | | | | | | | | |
|---|---|---|---|---|---|---|---|---|---|---|
| | Dental Floss | | Base Coating | | Particulate Overcoating | | | | | |
| | Micromesh | | Base | | | | | | Particulate | |
| | Dental | Denier | Coat Formula | Base Coat | | Base Coat & | Particulate | Particulate | Abrasive | |
| | Floss | (grams/ | with | Load | Particulate | Particulate Load | Abrasive Load | Abrasive % | % of Total | |
| Ex No. | Type | yd) | Chlorhexidine | (mg/yd) | Type | (mg/yd) | (mg/yd) | of total load | Device | PAF |
| Micromesh flattened | | | | | | | | | | |
| fibrillated 300d | | | | | | | | | | |
| 29 | Softmint | 0.046 | Ex 28 | 0.0552 | Granular DCP | 0.069 | 0.0138 | 20.0 | 12.0 | 2.0 |
| 30 | Softmint | 0.044 | Ex 28 | 0.057 | Silica | 0.0755 | 0.0185 | | | 2.6 |
| 31 | Softmint | 0.04 | Ex 28 | 0.0512 | 3F Pumice | 0.0788 | 0.0276 | 35.0 | 23.2 | 3.4 |

Comparing the particulate abrasive overcoated versions of coated micromesh dental flosses, as described in Examples 29 to 31, with the corresponding coated micromesh flosses without the particulate abrasive overcoating indicates a dramatic improvement in the “hand” of the particulate abrasive overcoated version, as well as in the perception that the particulate abrasive overcoated micromesh dental floss is “working”. See PAF values. These improvements are considered substantial and relevant and contribute to the overall enhanced perceived value of these particulate abrasive overcoated versions of micromesh dental flosses, compared to the commercial versions without these overcoatings.

Comparing particulate abrasive overcoated versions of micromesh floss with J&J Waxed Mint multifilament dental flosses and J&J Whitening Dental Tape, indicates the particulate abrasive overcoated versions of these two micromesh dental flosses are preferred over J&J Whitening Dental Floss and J&J Waxed Mint Floss. This preference is in part attributed to the ease of use and ease of insertion indicated for the particulate abrasive overcoated micromesh dental flosses along with the perception that these particulate abrasive overcoated versions are “working” as further indicated by the PAF values.

A particularly preferred embodiment of the present invention is the enhanced perceived value imparted to a wide range of coated micromesh dental flosses with very modest increases in cost-of-goods. This enhanced perceived value can be achieved by the addition of a modest priced particulate abrasive overcoating using an overcoating operation that can be installed in-line with current waxing and/or coating operations.

Commercial, coated, micromesh dental flosses such as described in Examples 29 through 31 in Table 4 can be further improved beyond the “it's working” perception, which is indicated by recorded PAF values. That is, a second overcoating with a saliva soluble particulate containing flavor, mouth feel agents, etc., can be imbedded into the saliva soluble base coating using a second separate fluidized bed and nozzle means to imbed this particulate into the liquid base coating before the micromesh floss enters the coating zone.

TABLE 4
Coat Micromesh Dental Floss Overcoated with Particulate Abrasive on Saliva Soluble Particulate

| | | | Particulate | | | Overcoatings Saliva | |
|---|---|---|---|---|---|---|---|
| | Micromesh | Base Coating | Abrasive Type & | | | Soluble Particulate Type | Projected Impact of |
| Ex. | Dental Floss & | Type & Load | Load | Projected | Projected | & Load | Saliva Soluble |
| No. | Denier | (mg/yd) | (in mg/yd) | PAF | IRF | (in mg/yd) | Particulate |
| 32 | UHMWPE | Saliva Soluble | pumice | 3.4 | 96 | PEG 3350/flavor | 3 X over |
| | 415 | (62) | (21) | | | (14) | wax flavor |
| 33 | UHMWPE | Saliva Soluble | pumice | 3.2 | 98 | PEG 3350/flavor | 4 X over |
| | 415 | (58) | (14) | | | (18) | wax flavor |
| 34 | UHMWPE | Saliva Soluble | silica | 2.8 | 97 | PEG 3350/flavor | 2 X over |
| | 421 | (72) | (16) | | | (12) | wax flavor |
| 35 | UHMWPE | Saliva Soluble | pumice | 3.5 | 92 | PEG 3350/flavor | 2 X over |
| | 421 | (66) | (22) | | | (14) | wax flavor |
| 36 | UHMWPE | Saliva Soluble | pumice | 3.0 | 96 | PEG 3350/flavor | 3 X over |
| | 418 | (64) | (14) | | | (17) | wax flavor |

Dental experts have been saying for years . . . “There's simply NO SUBSTITUTE for flossing.” Dental experts now overwhelmingly also agree that if you suffer from chronic diseases, including diabetes and heart disease, flossing is a critical preventative step necessary for removing plaque from between the teeth and below the gum line. Plaque (biofilm) buildup causes gum disease (gingivitis), which affects some two-thirds of the U.S. population, while advanced-stage gum disease (periodontal disease) is the leading cause of tooth loss in American adults and affects between ten and fifteen percent of the U.S. population.

The emergence of “Biofilms” as a key element in future oral health care was confirmed with the presentation of over 25 “Biofilm” abstracts at the IADR/AARR/CADR 83^{rd} General Session, March 9-12, 2005, at the Baltimore, Maryland Convention Center.

### Dental Flossing and Chronic Diseases

The present invention is directed to a medical device and associated methods for controlling the influence of oral cavity based microbiological burden on chronic diseases such as Type II diabetes and heart disease. The medical device removes and disrupts biofilm while delivering an antimicrobial ingredient, such as chlorhexidine digluconate, to residual interproximal, supragingival and subgingival sites. The combination product has the primary intended purpose of fulfilling an interproximal device function; namely, physically: removing and disrupting biofilm (plaque) attached to tooth surfaces and simultaneously topically treating, controlling and disrupting biofilm not totally physically removed from tooth surfaces by flossing, with a topically applied antimicrobial, such as chlorhexidine digluconate (chlorhexidine); thereby establishing and maintaining periostasis . . . and avoiding the exacerbation of various chronic diseases by the microbiological burden associated with biofilms.

The device of the present invention is:
(1) a compression coated monofilament interproximal medical device designed to physically remove and disrupt biofilm;
(2) a chlorhexidine delivery system suitable for maintaining periostasis by patient self-treatment, once daily;
(3) a SOFT ABRASIVES® delivery system suitable, during flossing, to physically remove and disrupt biofilms, stains, etc.; and
(4) a fibrillated monofilament medical device designed to physically entrap and remove loosened biofilm, food particles, materia alba and debris from between teeth.

This biofilm-responsive chlorhexidine/SOFT ABRASIVES® delivery system and associated method are designed for maintaining periostasis in cases of mild to moderate biofilm buildup as indicated by bleeding sites, i.e., gingivitis and gingival detachment up to 3 mm, and as an adjunct to periodic professional prophylaxis and/or other professional treatments, including root planing and scaling.

See FIGS. 16 through 18 for a schematic illustration of interproximal, supragingival and subgingival delivery of the ingredient, chlorhexidine, to residual biofilm, while simultaneously physically removing and disrupting biofilm attached to interproximal, supragingival and subgingival tooth surfaces, thereby maintaining periostasis.

To maintain periostasis, during each flossing, the delivery system, which comprises an 18-21 inch piece of coated dental tape with an overcoating of SOFT ABRASIVES®, releases about 80 mg/yd of a saliva soluble coating containing 3.8 mg/yd of chlorhexidine and about 4 mg/yd of SOFT ABRASIVES® overcoating to from between about 10 and about 36 interproximal and subgingival sites throughout the mouth. Thus, the average delivery of chlorhexidine per interproximal and/or subgingival site is from between about 0.38 and about 0.001 mg, with a total of 3.8 mg/yd being delivered to the oral cavity during each flossing.

The devices of the present invention are particularly adapted for maintaining periostasis in cases of biofilm buildup accompanied by bleeding sites, i.e., gingivitis and gingival detachment, and as a patient periostasis self-treatment adjunct to professional procedures. The devices of the present invention provides several site-specific modes of action, i.e.,:
(a) Physical removal and disruption of biofilm via flossing;
(b) Antimicrobial topical treatment of microorganisms associated with residual biofilms not removed by flossing via delivery of the ingredient, chlorhexidine digluconate, to various interproximal biofilm sites remaining supragingivally and subgingivally, after flossing;
(c) Physical abrasion of chlorhexidine-stained pellicle with SOFT ABRASIVES® released from the dental tape during flossing; and
(d) Physical entrapment and removal of loosened biofilm, debris, food particles and materia alba from between teeth.

Thus, when flossing with fibrillated substrates, combined with SOFT ABRASIVES® of the present invention, does not physically remove the biofilm totally from a specific site, the simultaneous topical delivery of the antimicrobial ingredient, chlorhexidine digluconate, to the disrupted biofilm remaining at this site, assures that the residual biofilm microorganisms remaining are topically treated, controlled and disrupted by the antimicrobial, chlorhexidine digluconate, thereby maintaining periostasis. Thus, regular flossing with the devices of the present invention holds the microbiological burden in-check (periostasis), thereby controlling its influence on various chronic conditions of at risk adults, such as Type II diabetes heart disease, etc.

Gingivitis is a microbe-mediated gingival disease that can cause periodontitis. The main cause of chronic gingivitis is bacterial plaque (biofilm) resulting from the colonization of bacteria on tooth surfaces and under the gingival margin. Rinsing and tooth brushing are ineffective in physically removing biofilms from hard-to-reach interproximal, supragingival and subgingival tooth surfaces. Only regular and effective use of the biofilm-responsive interproximal devices of the present invention can physically remove and disrupt biofilms from these surfaces and simultaneously control residual biofilm remaining after flossing to maintain periostasis between professional prophylaxes and/or other professional treatments, including scaling and root planing. The micro-organisms in biofilms produce toxins, metabolic end products and enzymes that invade the gums causing inflammation, which is characterized by swollen, bleeding gums . . . gingivitis. Gingivitis can lead to loss of gingival-tooth attachment and the formation of periodontal pockets . . . periodontitis. Left untreated, periodontitis can lead to progressive loss of periodontal ligaments, bone resorption and tooth loss. Chlorhexidine and other broad spectrum antimicrobials have been used as part of various treatment regimens that include the periodic manual physical removal of biofilms by oral care professionals, such as prophylaxis procedures, scaling and root planing.

Chlorhexidine was chosen as the antimicrobial of choice for inclusion in the devices and methods of the present invention to maintain periostasis, because:
1. of its history of lack of bacterial resistance;
2. chlorhexidine is currently approved in the U.S. for use orally as an Rx, 0.12%, topical, oral rinse (Peridex® and others) with anti-biofilm and anti-gingivitis claims;
3. chlorhexidine is currently approved in the U.S. for use orally as a Rx 2.5 mg site-specific, professional, topical treatment for periodontal pockets greater than 5 mm in depth (PerioChip®), where the chlorhexidine is released from the bioabsorbable chip over 7 to 10 days;
4. chlorhexidine remains stable in the saliva soluble coating on the devices of the present invention prior to flossing and is topically released at a rate of 3.8 mg/yd interproximally, supragingivally and subgingivally during each flossing;
5. chlorhexidine is a positively charged molecule which, when released into the oral cavity, indicates substantivity, i.e.,:
   (a) it is readily attracted to negatively charged bacterial cells attached to teeth, and
   (b) it has a strong affinity for mucous membrane; and
6. The substantivity of chlorhexidine to various oral cavity surfaces continues for up to about 8 hours.

The molecular description of chlorhexidine digluconate (chlorhexidine) is set forth in the USP Dictionary. Specifically, chlorhexidine is 1,1-N-hexamethylene bis(5-(p-chlorophenyl biguanide) di-D-gluconate, a cationic bisbiguanide. Molecular formula: C₃₄H₅₄Cl₂N₁₀O₁₄. Molecular Weight: 897.77; having the following structure:

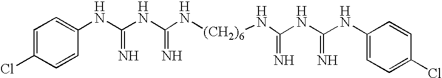

Chlorhexidine is a strong base, practically insoluble in water. Solubility is dependent on the salt form. Chlorhexidine digluconate is the most soluble form of chlorhexidine.

The antimicrobial spectrum of activity of chlorhexidine includes vegatative gram-positive and gram negative bacteria inclusive of vegatative anaerobes. It is inactive against bacterial spores except at elevated temperatures. Chlorhexidine has antifingal activity with this activity being greater against the yeast forms than the mold forms. The level of activity varies with the species of the fungi. As is the case with bacterial spores, chlorhexidine is inactive against fungal spores. Chlorhexidine has been shown to have clinically relevant activity against those bacteria which have been associated with gingivitis.

Chlorhexidine is a broad spectrum antimicrobial agent. The mechanism by which chlorhexidine exerts antimicrobial effects in not well defined, but may include damage to the bacterial cell wall through action as a surfactant. Various species of bacteria are thought to be involved in the pathogenesis of gingivitis. It is hypothesized that the therapeutic effects of the devices of the present invention are mediated through effects on biofilm residue remaining after flossing designed to remove, disrupt and control biofilm. The simultaneous interproximal and subgingival delivery of chlorhexidine gluconate to residual disrupted biofilm sites by flossing helps compensate for less than total removal of biofilms.

At low concentrations (approximately<100 μg/mL) chlorhexidine tends to be bacteriostatic while at higher concentrations it is bactericidal. The mechanism of bacteriostatis is not well understood. The bactericidal concentrations vary from genus to genus of microorganisms and within the genus from species to species.

The main site of action of chlorhexidine is the cellular membrane of bacteria and fungi and the lipophilic envelope of viruses. This activity against the cellular membrane results in dissolution of the membrane with resulting leakage of the cytoplasmic content. In the case of chlorhexidine-induced leakage of intracellular material from Escherichia coli and Staphylococcus aureus a diphasic leakage/concentration pattern is found. The first part of the pattern (kill curve) shows increasing leakage of cytoplasm as the concentration of chlorhexidine increases. The second part of the curve shows that at higher concentrations the leakage actually slows. This is due to the fact that the chlorhexidine causes a coagulation of the cytoplasmic protein and this coagulation tends to slow down the flow of the cytoplasmic content from the affected cell. Bacteriostatic concentrations of the compound do not cause leakage of cytoplasmic material. At bacteriostatic concentrations enzyme activity associated with transport activities across the cell membrane are believed to be inhibited. The rapid activity of chlorhexidine against bacteria is partially attributed to the fact that chlorhexidine is a positively charged molecule which is readily attracted to the negatively charged bacterial cell.

A “depathogenizing” effect of chlorhexidine has been described in the literature. The term relates to the phenomenon that sublethal levels of chemicals alter or damage bacterial cells in such a way to reduce their ability to initiate the disease process. Holloway showed this effect with chlorhexidine in a mouse peritonitis model. Pathogenic strains of Escherichia coli and Klebsiella aerogenes treated with sublethal concentrations of chlorhexidine were shown to be less capable of causing infection in the mouse. This work was later confirmed by Rotter. Minhas et. al. has shown that sublethal concentrations of chlorhexidine significantly inhibit the production of trypsin-like proteases in Porphyromonas (Bacteriodes) gingivalis. The significance of these findings to the periodontal disease process is not specifically known. However, the potential exists that while organisms may be culturable from diseased sites their ability to cause disease is reduced. A possible measure of this would be the return to health of the diseased area and not the absence or the presence of periodontal pathogens.

Chlorhexidine was first synthesized in 1950 and shown at that time to have antibacterial and antifungal properties, a strong affinity for skin and mucous membranes, and minimal toxicity. Shortly thereafter it was introduced into the market as an antiseptic for application to skin, wounds, and mucous membranes. In addition, it is used as a preservative for ophthalmic solutions and as a disinfectant. Despite the use of chlorhexidine as an antimicrobial in a variety of products for over 50 years, no conclusive evidence exists in the literature that microorganisms have developed resistance to it.

In the dental profession chlorhexidine has been advocated to be used to: prevent caries, inhibit the development of plaque and gingivitis and treat dental infections for over 25 years. The effects of chlorhexidine on the development of plaque has been studied extensively. Many studies have looked for the development of resistance to chlorhexidine in plaque bacteria after use of chlorhexidine for as long as two years and while there were slight sporadic changes in the oral flora susceptibility to chlorhexidine, long term resistance was not found. The bacteria isolated from the plaque were also shown to maintain there susceptibility to antibiotics after prolonged use of chlorhexidine. Studies using chlorhexidine to treat periodontal disease that have looked at the development of chlorhexidine resistant bacteria or bacteria resistant to unrelated chemicals or antibiotics have not conclusively identified this as a matter of concern. Chlorhexidine has been reported for treating gingivitis using a broad array of chlorhexidine therapies including rinses, acrylic strips, subgingival irrigations, etc.

### Periostasis

The literature indicates that regular brushing and flossing removes, disrupts and controls, on average, about 70% of the total biofilms deposited on tooth surfaces. Sporadic and/or infrequent brushing and flossing removes, disrupts and controls even less biofilm.

Total removal of biofilm requires a periodic, complete professional cleaning (prophylaxis).

Disruption and control of the bacteria associated with residual “hard-to-remove” biofilms requires regular topical site-specific administration of a substantive antimicrobial by means of an interproximal delivery device of the present invention. The resultant, repetitive, antimicrobial, topical treatment neutralizes the potential of the residual biofilm bacteria, not physically removed by flossing, to exacerbate gingivitis, detached gingiva, gingival bleeding, etc. . . . between professional cleanings. Thus, regular physical removal, disruption and control of biofilms by daily flossing is enhanced by the simultaneous, chemotherapeutic, antimicrobial disruption and control of those “hard-to-reach”, residual biofilms not physically removed by flossing; thereby maintaining a stable gingival condition . . . between professional visits, which is described herein as “periostasis.”

This regular, site-specific, chemotherapeutic disruption and control of hard-to-reach biofilms that are not thoroughly removed by brushing and flossing, and which are not reached by rinsing, provides at risk patients a stabilized, gingival condition, periostasis. Periostasis is key to at risk patients minimizing the exacerbation potential of existing gingivitis, detached gingiva, gingival bleeding, etc.

Periostasis defines a stabilized gingival condition identified with at risk adults, where biofilm-triggered gum disease, including: gingival detachment, bleeding gums, etc., as well as biofilm bacteria-based exacerbation of chronic systemic conditions are abated between professional visits.

Regular flossing with the device of the present invention physically removes and disrupts biofilms on those interproximal, supragingival and subgingival surfaces that cannot be reached by brushing and/or rinsing. Regular flossing with the devices of the present invention helps control gum disease, including: gingival detachment among at risk adults between professional visits. Regular brushing and flossing are estimated to remove, disrupt and control up to about 70% of all biofilms. Only professional prophylaxis removes substantially all biofilms. The devices of the present invention focus on maintaining a stable gingival condition, periostasis for at risk adults, between professional visits.

During flossing with the devices of the present invention by at risk adults, the simultaneous release from the floss of the substantive antimicrobial, chlorhexidine digluconate, chemotherapeutically augments the physical removal and disruption of biofilms achieved by flossing with SOFT ABRASIVES® while also helping to disrupt, control and neutralize those residual biofilm-based bacteria associated with gum disease . . . thereby establishing periostasis. See FIGS. 16 through 18.

In addition to the control of gingivitis, adult Type II diabetes patients with at least up to 3 mm gingival detachment, using the devices of the present invention, are expected to maintain periostasis and indicate a clinically significant reduction in and/or control of glycated hemoglobin levels.

The devices of the present invention offer an opportunity to maintain periostasis, along with reduced HbA levels of diabetes patients after these patients have undergone successful professional treatment for periodontal disease, including administration of systemic doxycycline.

Grossi, et. al., in J. Periodontol. 1997; 68:713-719, reported that after two weeks of treatment with a regimen of systemic doxycycline, combined with ultrasonic bactericidal curettage (UBC) employing continuous irrigation with an antimicrobial solution, there was an impressive improvement of oral health. For example, at 3 and 6 months after treatment, significant reduction in: plaque scores, gingival scores and mean probing depth were indicated. An antibacterial mechanism of action seems to have been indicated by the absence of detectable p. gingivalis. See also other Grossi and/or Genco key references.

These clinical improvements in periodontal health were associated with a significant reduction in levels of glycated hemoglobin (HbA) for up to three months after treatment, impacting health issues well beyond oral health.

Unfortunately, within 12 months after treatment, the HbA scores returned to baseline. Perhaps this disappointment is not terribly surprisingly, given the opportunity for re-infection in the not-completely-healed gingival pockets.

The present invention suggests the improvement in HbA levels, reported by Grossi, et. al., could be maintained by following up this “professional periodontal treatment” with a “maintenance” antigingivitis, patient self-treatment, where periostasis is maintained. This follow-up “periostasis maintenance” treatment calls for daily flossing with the devices of the present invention.

This floss has been demonstrated to deliver significant quantities of antimicrobially-active CHX to interproximal sites. (Data indicates the Rx floss delivers at least 3× the CHX interproximally compared to commercial CHX rinse.)

Ideally, the proposed patient “periostasis maintenance” self-treatment with the floss of the present invention would start immediately following the professional treatment detailed in the Grossi, et. al., study. Ideally, the “periostasis maintenance” treatment should begin immediately after the professional treatment and no later than three months after conclusion of the professional treatment. It is proposed, if the significant periodontal improvement reported by Grossi, et. al., could be at least maintained and, perhaps even improved further, with this daily flossing, patient self-treatment “periostasis maintenance” program.

The Genco/Grossi publications from the School of Dentistry Department of Oral Biology of the State University of New York at Buffalo report that systemic antibiotic treatment with Periostat®, combined with professional scaling and root planing, controls levels of bacteria that cause gum disease, as well as C-reactive protein and fibrinogen protein levels in periodontitis patients. Once, under control, these bacteria levels can be maintained by patient daily self-treatment with the devices of the present invention, thereby maintaining periostasis.

Presuming that: (1) heart disease has a substantial inflammatory component, and (2) carotid artery thickness, an indicator of atherosclerosis, is dependent upon the level of bacteria in the mouth that causes gum disease; the present invention is directed to maintaining periostasis with at risk atherosclerosis patients with periodontal disease using the biofilm-responsive, antimicrobial dental floss of the present invention, where the treatment comprises once daily removal and disruption of interproximal biofilms and the simultaneous interproximal, site-specific delivery of the antimicrobial, chlorhexidine digluconate, to residual biofilm not removed by flossing.

This removal and disruption of biofilms and simultaneous antimicrobial, site-specific, topical treatment of residual biofilms is expected to indicate a reduction in gingival bleeding (and a corresponding reduction in periodontal bacteria burden and maintenance of periostasis), along with control of carotid artery intima-media thickness associated with increased risk of heart disease. See Columbia University Medical School publication by Desvarieux, et. al., in Circulation 2005.

The proposed topical, antimicrobial, patient self-treatment adjunct to the professional treatment of gum disease reported by Genco/Grossi using the device of the present invention is expected to also maintain periostasis for persons with moderate to high risk of atherosclerosis. This may prove to be a critical health care, prevention step for this at risk population.

Findings of M. Desvarieux, et. al., in Circulation, 2005; 111:576-582, strengthen the hypothesis that: Oral infections may contribute to cardiovascular disease morbidity and bolster the supposition that accelerated atherosclerosis development is a possible mechanism connecting chronic infections and cardiovascular disease. Specifically, “Periodontal infections predispose to accelerated progression of carotid atherosclerosis and incidence of stroke, myocardial infarction and cardiovascular disease death.”

Among the microbes assayed from the subgingival environment adjacent to selected teeth . . . carotid intima-media thickness (IMT) correlated cross-sectionally with:
(1) the cumulative microbiological burden in the periodontium,
(2) specifically, the organisms causally associated with periodontal disease, and
(3) the microbial dominance of these causal organisms in relation to other organisms of the ecological niche.

It is proposed that once daily flossing with the floss of the present invention will control interproximally, supragingivally and subgingivally:
(a) the microbiological burden in the periodontium,
(b) the specific organisms causally associated with periodontal disease,
(c) the microbial dominance of causal organisms in relation to other organisms, and
(d) the buildup of biofilms;
   thereby achieving periostasis and reducing and perhaps reversing atherosclerotic damage through selective control of pathogenic periodontal bacteria by a combination of physical removal, disruption and control of interproximal and subgingival biofilms and their associated pathogenic bacteria.

It appears that daily flossing with the floss of the present invention could play a key public health role by reducing and perhaps reversing atherosclerotic damage and maintain periostasis through:
Physical removal, disruption and control of interproximal and subgingival biofilms
Topical control of pathogenic periodontal bacteria associated with interproximal and subgingival biofilms, and
SOFT ABRASIVES® control of chlorhexidine staining of those tooth surfaces associated with topical treatment with chlorhexidine.

The present invention has been described in detail, including the preferred embodiments thereof. However, it will be appreciated that those skilled in the art, upon consideration of the present disclosure, may make modifications and/or improvements on this invention and still be within the scope and spirit of this invention as set forth in the following claims.

What is claimed is:

1. An interproximal device suitable for use between professional oral care treatments of various biofilms associated with exacerbating various chronic conditions selected from the group consisting of: Type II diabetes mellitus, atherosclerosis, heart disease, osteoporosis, HIV, myocardial infarction, and combinations thereof comprising a fibrillated, high molecular weight polyethylene tape that is compression coated with a saliva soluble coating containing an antimicrobial and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba.

2. A method for treating at risk adults between professional oral care treatments of various biofilms associated with exacerbating various chronic conditions selected from the group consisting of: Type II diabetes mellitus, atherosclerosis, heart disease, osteoporosis, HIV, myocardial infarction, and combinations thereof, comprising flossing regularly with an interproximal device comprising a fibrillated, high molecular weight polyethylene tape that is compression coated with a saliva soluble coating containing a substantive antimicrobial and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba,
and removing said spent tape from interproximal spaces.

3. An interproximal device suitable for use between professional oral care treatments for controlling biofilms associated with exacerbating glycated hemoglobin levels of Type II diabetics, comprising flossing regularly with an interproximal device comprising a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba.

4. A method of controlling biofilm-supported glycated hemoglobin level of Type II diabetics, adapted for use between professional oral care treatments, comprising flossing regularly with an interproximal device comprising a fibrillated, high molecular weight polyethylene tape that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls antimicrobial-based tooth staining, and
(d) physically entraps: loosened biofilm, debris, food particles and materia alba,
and removing said spent device from said interproximal spaces.

5. An interproximal device for use between professional oral care treatments suitable for reducing and controlling biofilms associated with exacerbating glycated hemoglobin levels of Type II diabetics, comprising a fibrillated, high molecular weight polyethylene dental device that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls chlorhexidine-based tooth staining, and
(d) physically entraps: loosened biofilm, debris, food particles and materia alba.

6. A method of reducing and controlling biofilm-supported glycated hemoglobin levels of Type II diabetics, adapted for use between professional oral care treatments, comprising flossing regularly with an interproximal device comprising a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls chlorhexidine-based tooth staining, and
(d) physically entraps: loosened biofilm, debris, food particles and materia alba,
and removing said spent tape from interproximal spaces.

7. An interproximal device for use between professional oral care treatments, suitable for controlling biofilms associated with carotid artery intima media thickness and increased risk of heart disease, comprising a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said antimicrobial contained in said saliva soluble coating onto said residual biofilms, thereby maintaining periostasis,
(c) controls chlorhexidine-based tooth staining, and
(d) physically entraps: loosened biofilm, debris, food particles and materia alba.

8. A method of controlling biofilms associated with exacerbating carotid artery intima media thickness and increased risk of heart disease among at risk adults, adapted for use between professional oral care treatments, comprising flossing regularly with an interproximal device comprising a fibrillated, high molecular weight polyethylene dental device that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms, thereby maintaining periostasis,
(c) controls chlorhexidine-based tooth staining, and
(d) physically entraps: loosened biofilm, debris, food particles and materia alba,
and removing said spent tape from interproximal spaces.

9. An interproximal device suitable for use between professional oral care treatments for controlling biofilms associated with exacerbating low birth weight babies, comprising a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal and subgingival biofilms remaining after flossing by topically releasing said chlorhexidine digluconate onto said residual biofilms, thereby maintaining periostasis,
(c) controls chlorhexidine-based tooth staining, and
(d) physically entraps: loosened biofilm, debris, food particles and materia alba.

10. A method of controlling biofilms associated with exacerbating low birth weight babies, adapted for use between professional oral care treatments, comprising having pregnant mothers-to-be floss regularly with an interproximal device comprising a fibrillated, high molecular weight polyethylene tape that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms, thereby maintaining periostasis,
(c) controls chlorhexidine-based tooth staining, and
(d) physically entraps: loosened biofilm, debris, food particles and materia alba,
and removing said spent tape from interproximal spaces.

11. An method of treatment of Type II diabetes patients, adapted as an adjunct to professional, mechanical, periodontal therapy, comprising regular, topical, patient self-treatment of interproximal sites having a propensity for pathogenic re-infection by Gram-negative organisms; comprising flossing with an interproximal device comprising fibrillated, high molecular weight polyethylene tape that is compression coated with a saliva soluble coating containing a Gram-negative responsive antimicrobial and overcoated with soft abrasives, wherein during flossing, said device:
(a) physically removes and disrupts interproximal biofilm;
(b) chemotherapeutically disrupts and controls Gram-negative organisms present in residual interproximal, supragingival and subgingival biofilms remaining after flossing, by topically releasing said antimicrobial contained in said saliva soluble coating onto said residual biofilm, thereby maintaining periostasis;
(c) controls antimicrobial-based tooth staining; and
(d) physically entraps: loosened biofilms, debris, food particles and materia alba,
and removing said spent tape from said interproximal sites.

12. A device suitable for maintaining periostasis in Type II diabetics, between professional oral care visits, comprising a fibrillated, high molecular weight polyethylene dental tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms,
(c) controls chlorhexidine-based tooth staining, and
(d) physically entraps: loosened biofilm, debris, food particles and materia alba.

13. A method of maintaining periostasis in Type II diabetics between professional oral care visits, comprising flossing regularly with a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms,
(c) controls chlorhexidine-based tooth staining, and
(d) physically entraps: loosened biofilm, debris, food particles and materia alba,
and removing said spent tape from interproximal spaces.

14. A method of maintaining periostasis in at risk heart disease patients between professional oral care visits, comprising flossing regularly with a fibrillated, high molecular weight polyethylene tape that is compression coated with a saliva soluble coating containing the antimicrobial, chlorhexidine digluconate, and overcoated with soft abrasives, wherein during flossing, said tape:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms,
(c) controls chlorhexidine-based tooth staining, and
(d) physically entraps: loosened biofilm, debris, food particles and materia alba.

15. A method of maintaining periostasis in at risk heart disease patients between professional oral care visits, comprising flossing regularly with a fibrillated, high molecular weight polyethylene tape that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival and subgingival biofilms remaining after flossing by topically releasing said chlorhexidine digluconate onto said residual biofilms,
(c) controls chlorhexidine-based tooth staining, and
(d) physically entraps: loosened biofilm, debris, food particles and materia alba,
and removing said spent tape from interproximal spaces.

16. An interproximal device suitable for:
(a) removing and disrupting biofilms,
(b) controlling interproximally, the subgingival and interproximal microbiological burden associated with residual biofilms remaining after flossing, thereby maintaining periostasis,
(c) removing, disrupting and controlling chlorhexidine-stained pellicle,
comprising a fibrillated, high molecular weight, polyethylene tape that is compression coated with a saliva soluble coating containing the antimicrobial, chlorhexidine digluconate, and overcoated with soft abrasives, wherein during flossing, said device:
(a) physically removes and disrupts interproximal biofilms,
(b) chemotherapeutically disrupts and controls the microbiological burden associated with residual interproximal, supragingival subgingival biofilms remaining after flossing by topically releasing said chlorhexidine digluconate onto said residual biofilms to maintain periostasis,
(c) removes, disrupts and controls chlorhexidine-based tooth staining, and
(d) physically entraps: loosened biofilm, debris, food particles and materia alba.
